(19) United States
(12) Patent Application Publication
(10) Pub. No.: US 2021/0032677 A1
(43) Pub. Date: Feb. 4, 2021

(54) Methods to Improve the Sequencing of Polynucleotides with Barcodes Using Circularisation and Truncation of Template

(71) Applicant: RootPath Genomics, Inc., Boston, MA (US)

(72) Inventors: Ely Porter, Medford, MA (US); Xi Chen, Newton, MA (US)

(73) Assignee: RootPath Genomics, Inc., Boston, MA (US)

(21) Appl. No.: 16/637,456

(22) PCT Filed: Aug. 9, 2018
(86) PCT No.: PCT/US2018/045893

§ 371 Date: May 4, 2020

Related U.S. Application Data
(60) Provisional application No. 62/543,612, filed on Aug. 10, 2017.

(51) Int. Cl. C12Q 1/6806; C12Q 1/6869; C12N 15/10

(52) U.S. Cl. CPC C12Q 1/6806; C12N 15/1065; C12Q 1/6869

(57) ABSTRACT

The application provides improved methods of analyzing biological particles and their constituents, including methods of generating truncated and barcoded nucleic acid molecules from at least two target polynucleotide sequences, each from distinct biological particles.

### FIELD

This relates to a method for generating truncated and barcoded nucleic acid molecules from at least two target polynucleotide sequences, each from distinct biological particles.

### BACKGROUND

Many methods have been developed to attach a barcode sequence to a target nucleic acid molecule. For example, inDrop™ (“indexing droplets,” Klein et al., Cell 161:1187-1201 (2015)), 10X platform from 10X Genomics (Zheng et al., Nat Commun 8: 14049 (2017)), and DropSeg™ (Macasco et al., Cell 161:1202 (2015)) (collectively referred to as “DropSeq-like methods”) can each attach a cell barcode and unique molecular index (also called “unique molecular identifier” or “UMI”) to cDNA. When combined with massively parallel sequencing (e.g., “NextGen Sequencing” or “NGS”), such barcoding methods can be immensely powerful in analyzing large numbers of biological samples (e.g., tens of thousands of individual cells). However, due to inherent limitations in some NGS technologies, often only sequence information for the portion of the nucleic acid in proximity to the barcode can be obtained using existing methods. For example, with Illumina, Inc.'s sequencers, library molecules with a long (e.g., >1,000 bp (base pairs)) insert tend to generate clusters with poor quality during bridge PCR. Thus, the DNA molecules to be sequenced are usually shortened to approximately 500 bp or less to accommodate this limitation. As a result, only sequences close to the barcode (e.g., within approximately 500 bp or less) have been able to be obtained using these methods. For this reason, DropSeq-like methods are considered 3′ sequencing techniques, because the barcode is attached to the 3′ end of the nucleic acid and the sequencing can only provide information on the region of approximately 500 bp or less to that 3′ end.

However, sequence distant from the barcode may be of interest. For example, in DropSeq-like methods the barcode is attached to the 3′ end of the mRNA molecule (or 5′ end of the first strand cDNA molecule); whereas one may be interested in learning about a splicing junction, a possible point mutation, or a hypervariable region several kilobases upstream in the mRNA molecule. Unfortunately, it is difficult to obtain such information using DropSeq-like methods.

We describe a series of circularization-based methods that generate sequencing libraries where sequence distant from the barcode can be brought to proximity with the barcode in linear DNA. These methods are collectively referred to as circularization-based DNA reorientation, or TeleLink™. The resultant DNA molecules can then be analyzed with NGS (e.g., using Illumina platforms) where both the barcode and the distant sequence can be read.

### SUMMARY

In accordance with the description, in one embodiment a method for generating truncated and barcoded nucleic acid molecules from at least two target polynucleotide sequences each from distinct biological particles comprises:
a. providing at least two heterogeneous pools of barcoded nucleic acid molecules each from a distinct biological particle, wherein each of the barcoded nucleic acid molecules comprise a target polynucleotide sequence and a barcode, wherein the barcode is unique to the distinct biological particle from which the barcoded nucleic acid molecule originated;
b. circularizing the barcoded nucleic acid molecules to obtain circular barcoded nucleic acid molecules; and
c. linearizing the circular barcoded nucleic acid molecules to obtain truncated and barcoded nucleic acid molecules comprising a truncated portion of the target polynucleotide sequence in the circular barcoded nucleic acid molecule and the barcode in the circular barcoded nucleic acid molecule.

In some embodiments, the method further comprises amplifying the truncated barcoded nucleic acid molecules to obtain a barcoded amplified product comprising the barcode and the portion of the target polynucleotide sequence.

In some embodiments, the truncated nucleic acid molecules are amplified using primers capable of binding to the primer-binding sites.

In some embodiments, the barcoded amplified product comprises a length of equal to or less than 500 base pairs.

In some embodiments, the barcoded nucleic acid molecules further comprise at least one primer binding site.

In some embodiments, the method further comprises introducing at least one primer-binding site to the truncated and barcoded nucleic acid molecules.

In some embodiments, the method further comprises truncating the target polynucleotide sequence before circularizing the barcoded nucleic acid molecules.

In some embodiments, the method further comprises ligating at least one additional domain to the truncated end of the barcoded nucleic acid molecule before circularizing the barcoded nucleic acid molecules.

In some embodiments, the method further comprises ligating at least one additional domain to barcoded nucleic acid molecules before circularizing the barcoded nucleic acid molecules.

In some embodiments, the barcoded nucleic acid molecule is DNA, RNA, or bisulfite-treated DNA.

In some embodiments, the target nucleic acid molecule is DNA.

In some embodiments, the target polynucleotide sequence is at least part of an engineered molecule that is used to engineer or probe the biological particle.

In some embodiments, the length of circular barcoded nucleic acid molecules is greater than 1 kb, 1.5 kb, 2 kb, 3 kb, 5 kb, or 10 kb.

In some embodiments, the distinct biological particles comprise cells, nuclei, or a cell cluster. In some embodiments, the biological particles are cells. In some embodiments, at least some of the cells are prokaryotic cells.

In some embodiments, at least some of the cells are eukaryotic cells.

In some embodiments, at least some of the cells are engineered with DNA, RNA or viral vectors that encode one or more biological agents that cause RNA-mediated gene knockdown, genome editing, transcriptional alteration, or epigenetic alteration.

In some embodiments, the one or more biological agents comprise one or more of siRNA, shRNA, miRNA, zinc finger domains, transcription activator-like effector (TALE), Cas9, RNA with CRISPR origin.

In some embodiments, the cell cluster comprises a T cell and an antigen presenting cell.

In some embodiments, the cell cluster comprises a cell that expresses an antigen-recognizing agent and a cell that expresses an antigen.

In some embodiments, the antigen-recognizing agent comprises an antigen-recognizing protein or an antigen-recognizing polynucleotide.

In some embodiments, the antigen-recognizing protein comprises an antibody, a functional antibody fragment, or a T cell receptor.

In some embodiments, the antigen is complexed with a major histocompatibility complex (MHC) molecule.

In some embodiments, the target polynucleotide sequence comprises a partial or complete T cell receptor sequence, or a partial or complete B cell receptor sequence.

In some embodiments, the target polynucleotide sequence comprises a mutation.

In some embodiments, the target polynucleotide sequence comprises a transcription start site.

In some embodiments, the target polynucleotide sequence comprises a splicing junction.

In some embodiments, a method for sequencing a target nucleic acid molecule comprises sequencing the barcoded amplified products.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate several embodiments and together with the description, serve to explain the principles described herein.

### BRIEF DESCRIPTION OF THE DRAWINGS

FIGS. 1A and 1B show a barcoded nucleic acid molecule and the modification thereof. FIG. 1A shows an exemplary structure of a barcoded nucleic acid molecule and FIG. 1B shows process by which a barcoded nucleic acid molecule is modified to be able to amplify an upstream sequence (109) between primer-binding sites P3 and P4. Barcoded nucleic acid molecule (101) is truncated at truncation site (102) to obtain molecule (103), optionally including additional domain X. Molecule (103) is circularized to obtain circular molecule (104). Circular molecule (104) is truncated at truncation site (105) and primer binding site P4 is added to obtain linear molecule (106) containing the upstream sequence (109). In FIGS. 1A and 1B, P1, P2, P3, and P4 represent primer binding sites, BC represents a barcode, the thin line (e.g., between P1 and BC in FIG. 1A) represents a sequence of interest, the whole zig-zag line (e.g., 102) and dotted zig-zag line (e.g., 108) perpendicular to the thin line represent truncation sites, the dashed arrow represents the upstream sequence (e.g., 109), and X represents an optional additional domain. Particularly, (108), (110), (111), (105) mark the same position on the sequence of interest. In addition, (109) and (112) mark the same upstream sequence that can be analyzed by sequencing.

FIG. 2 shows an exemplary circularization method of modified linear double-stranded DNA (dsDNA) (201). The thick black lines represent linear dsDNA having additional double-stranded domains (202) and (203) on each end. The 5′ end of top strand is modified with an optional biotin moiety (204) through a flexible linker, and the 5′ end of the bottom strand is modified with phosphate group (205). The arch (206) represents a solid surface for immobilization.

FIGS. 3A and 3B show a barcoded nucleic acid molecule and modification thereof. FIG. 3A shows an exemplary structure of a barcoded nucleic acid molecule and FIG. 3B shows a process by which a barcoded nucleic acid molecule is modified to be able to amplify an upstream sequence between primer-binding sites P3 and P4. Barcoded nucleic acid molecule (301) is circularized to obtain circular molecule (302). Circular molecule (302) is truncated at truncation site (303) and primer binding site P4 is added to obtain linear molecule (304) containing the upstream sequence. Molecule 304 can be amplified using primers targeting P3 and P4 to produce linear DNA (305). In FIGS. 3A and 3B, P1, P2, P3, and P4 represent primer binding sites, BC represents a barcode, the thin line (e.g., between P1 and BC on FIG. 3A) represents a sequence of interest, and (X) represents an optional additional domain.

FIG. 4 shows circularization-based nucleic acid reorientation (or TeleLink™) for a hypervariable region, such as a T-Cell Receptor (TCR) transcript or B-Cell Receptor (BCR) transcript using template-switching oligo (TSO). Reverse transcriptase (RT) primers (401) having the same cell barcode (CB) are hybridized to the poly-A tail of mRNA molecules (405) encoding the TCR/BCR, and undergo reverse transcription to copy the mRNA (Step 4.1). A TSO (402) with a few G bases can be paired with the C bases at the 3′ end of the first-strand cDNA (Step 4.2). The domain TS on the TSO can be cleaved (Step 4.3) and primers TS and DA can be used to amplify the first-strand cDNA (Step 4.4). The cDNA is circularized (Step 4.5) and the dashed lines represent a phosphodiester bond that link two segments of DNA. Primers (403 and 404) can be used to amplify the circular DNA (Step 4.6) to obtain dsDNA molecules. Additional PCR steps can be performed to attach additional domains to the dsDNA (Step 4.7). FIG. 4 can be considered an example of FIG. 3.

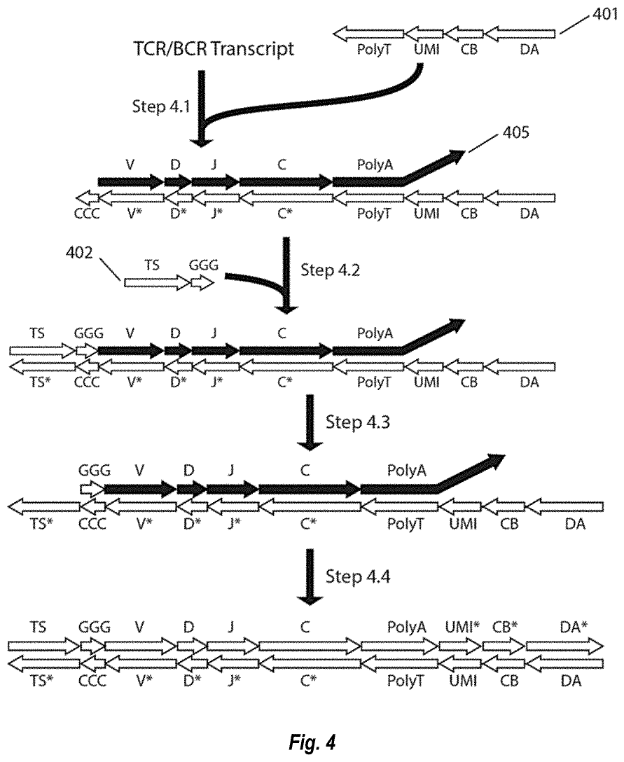

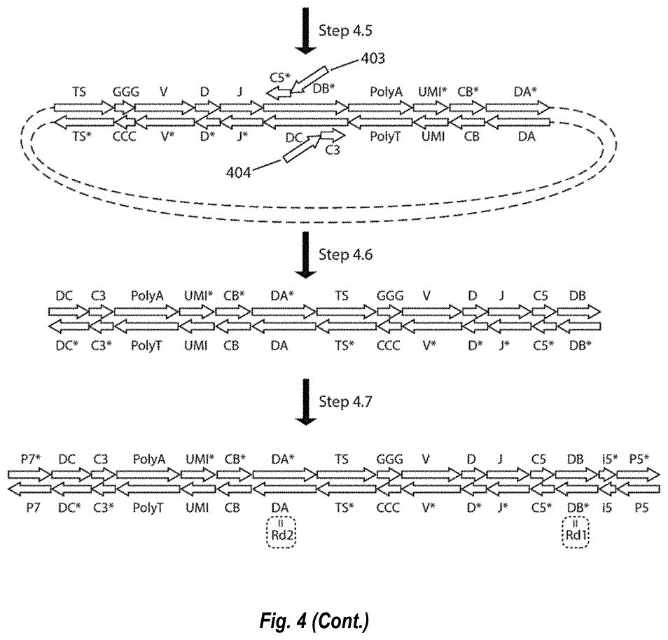

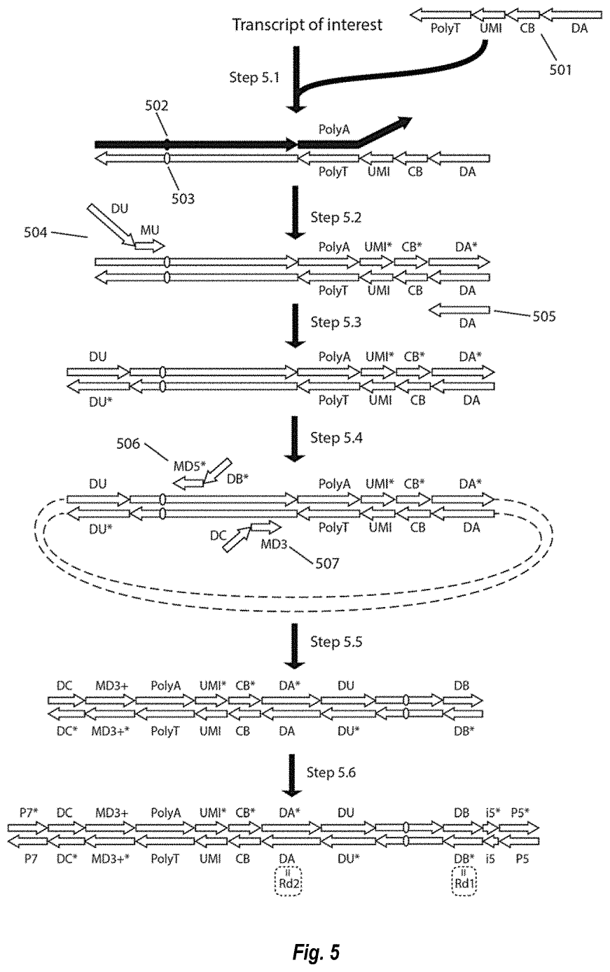

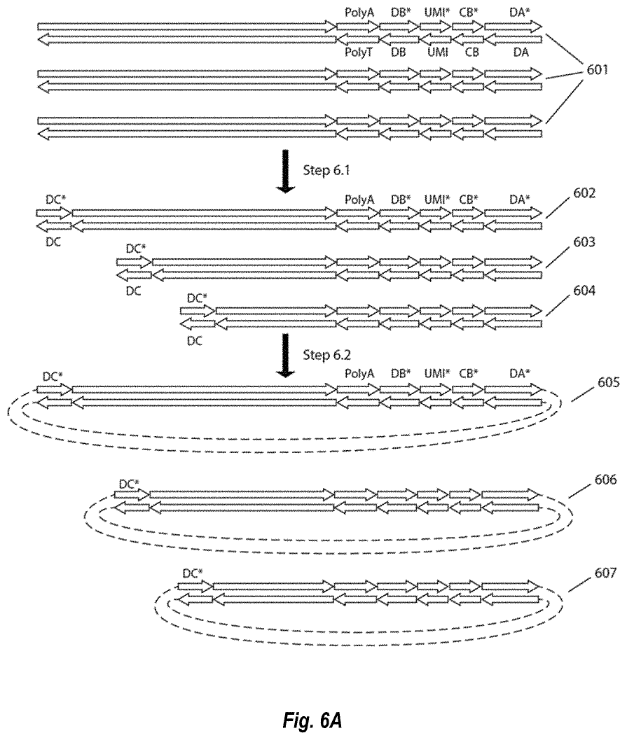

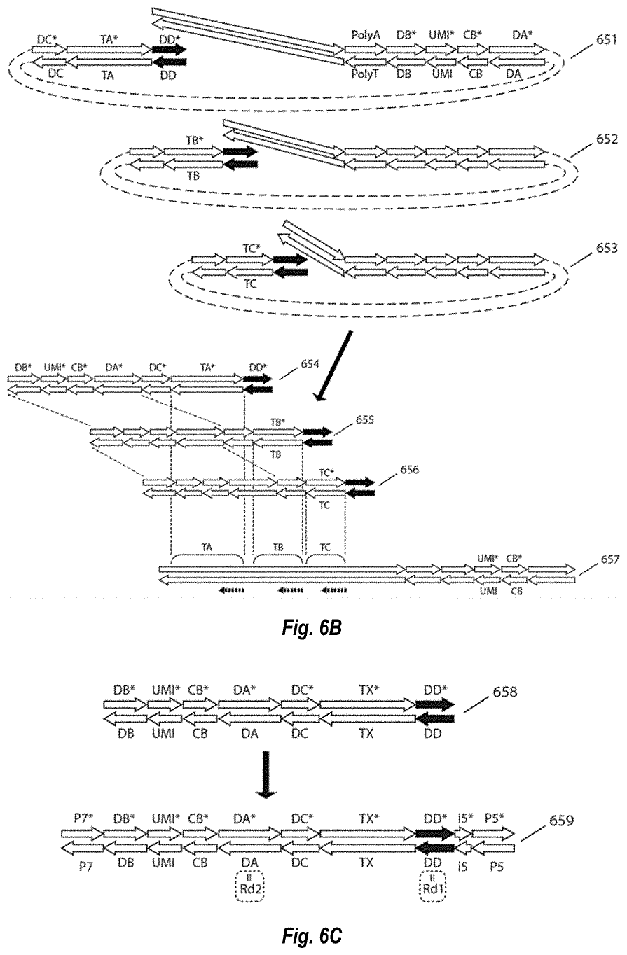

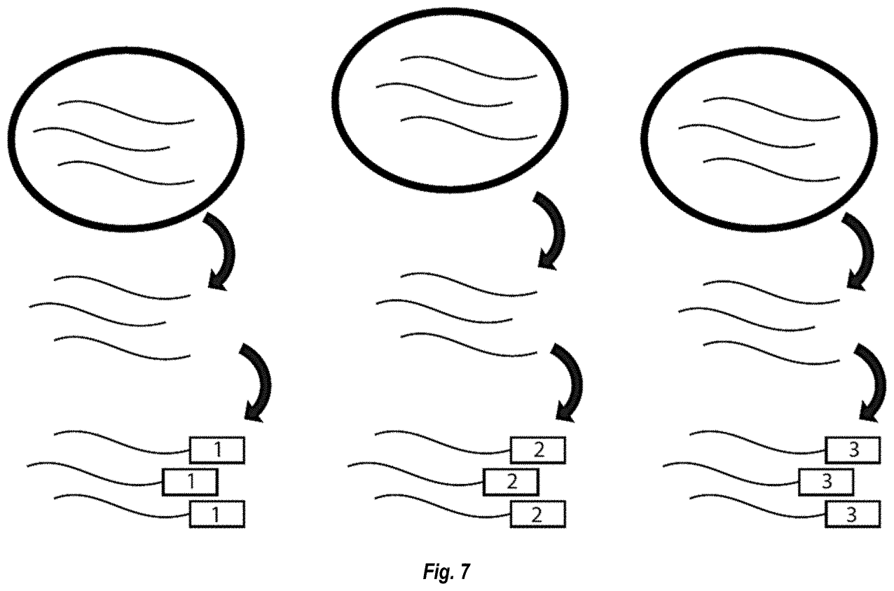

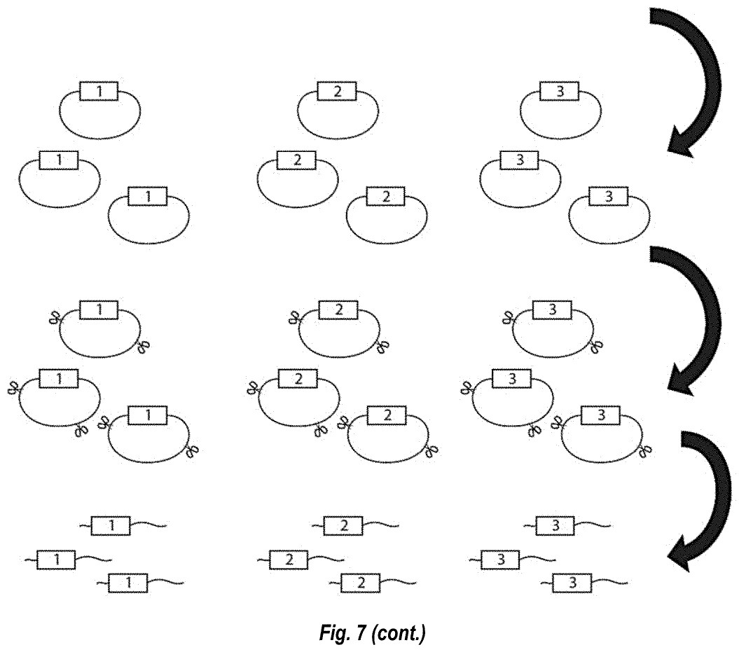

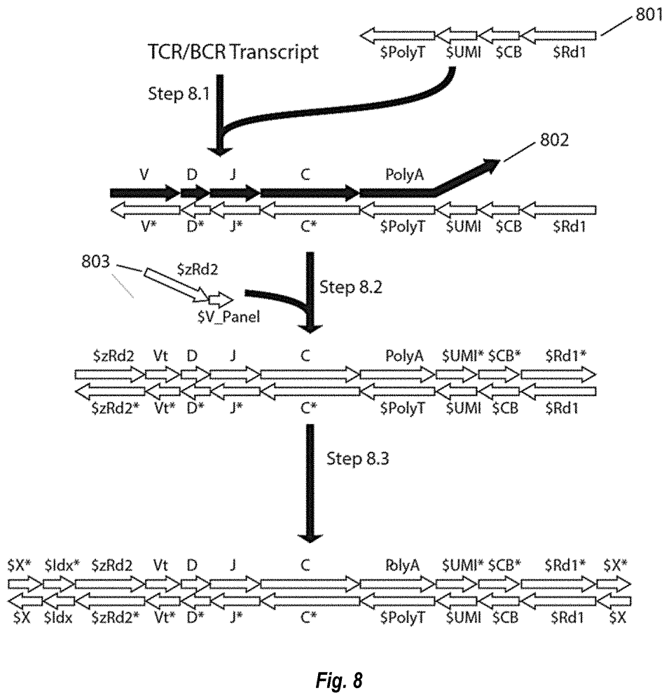

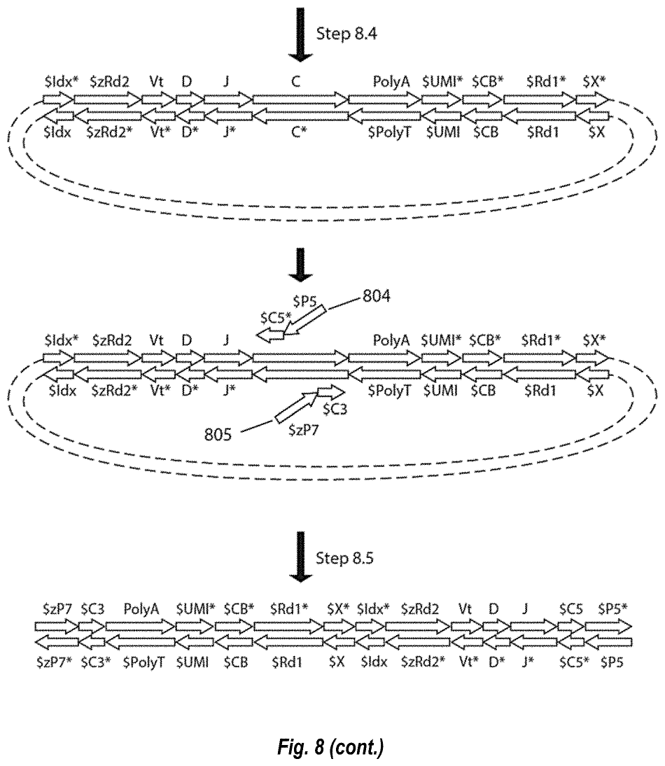

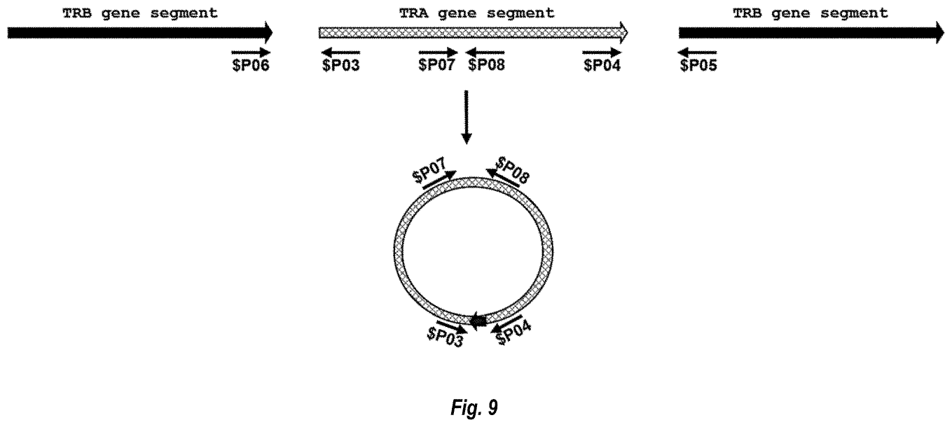

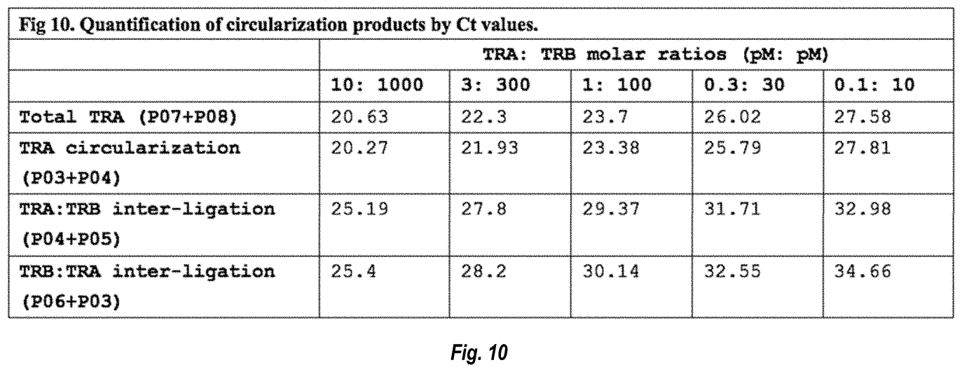

Table 1 discloses what each domain name in FIG. 4 represents.

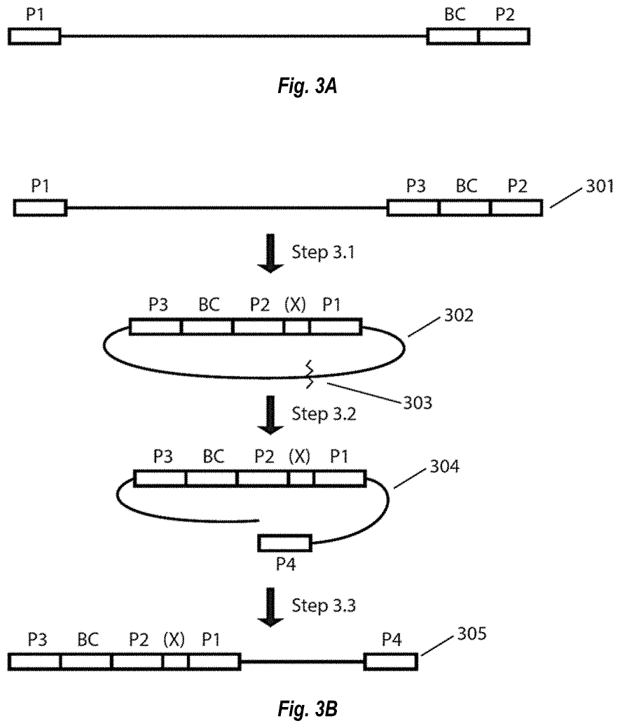

TABLE 1
Description of domains in FIG. 4

| Domain | | Functionally equivalent |
|---|---|---|
| name | Domain function | domain in FIG. 3 |
| DA | Primer binding site, suitable | P2 |
| | for circularization | |
| CB | Compartment or Cell barcode | Part of barcode (BC) |
| UMI | Unique Molecular Identifier | Part of barcode (BC) |
| PolyT | Reverse transcription primer | N/A, |
| | that binds poly A tail of | |
| | mRNA | |
| PolyA | Originated from part of the | N/A |
| | poly A tail on the mRNA | |
| V | V gene of TCR/BCR | Part of sequence of |
| | | interest |
| D | D gene of TCR/BCR | Part of sequence of |
| | | interest |
| J | J gene of TCR/BCR | Part of sequence of |
| | | interest |
| C | C gene of TCR/BCR | Part of sequence of |
| | | interest |
| TS | Template switching oligo, | P1 |
| | primer binding site, capable | |
| | of circularization | |
| C5 | Sequence close to the 5′ | Part of sequence of |
| | end of the C gene on the | interest, 3′ end of C5 |
| | TCR/BCR mRNA, primer | marks the truncation |
| | binding site | site 303 |
| C3 | Sequence close to the 3′ | P3 |
| | end of the C gene on the | |
| | TCR/BCR mRNA, primer | |
| | binding site | |
| DB | Adaptor sequence, possibly | P4 |
| | sequencing primer-binding | |
| | site | |
| DC | Adaptor sequence | N/A |
| Rd1, Rd2 | Exemplary sequences of DA | N/A |
| | and DB* domains (as in | |
| | Illumina platform) | |
| P5, P7 | Exemplary domains necessary | N/A |
| | to perform NGS | |
| i5 (index | Sample indices | N/A |
| read2 in | | |
| Illumina | | |
| platform) | | |

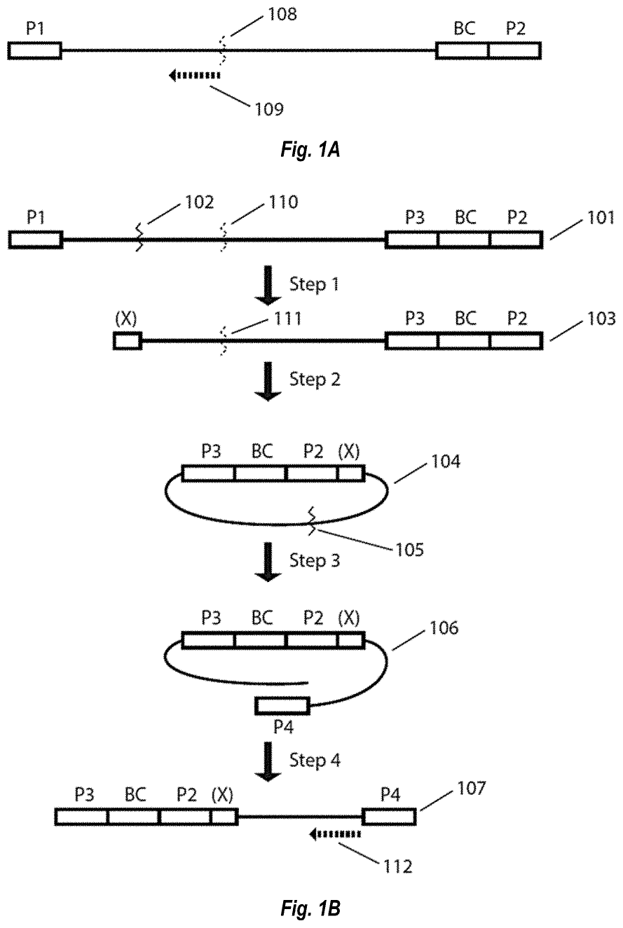

FIG. 5 shows another exemplary method of circularization-based nucleic acid reorientation (or TeleLink™). Barcoded RT primer (501) are hybridized to the poly-A tail of mRNA molecule (Step 5.1), which may contain a mutation (502). The mRNAs are reverse transcribed by the RT primer and reverse transcriptase to obtain first-strand cDNA that may carry a corresponding mutation (503). The mRNA:cDNA duplex may be converted to double-stranded DNA (Step 5.2). The cDNA can be PCR-amplified (Step 5.3) using a pair of primer (504 and 505), the PCR product can be circularized (Step 5.4). The circularized DNA may be further amplified using primers (506 and 507) (Step 5.5) to yield a linear dsDNA construct, the linear dsDNA can be further amplified with primers having additional domains to introduce new domains (e.g., P5, P7, and sample index domain i5) and the termini of the dsDNA (Step 5.6). FIG.5 can be considered an example of FIG. 1. Table 3 discloses what each domain name in FIG. 5 represents. Domain “MD3+” designates the sequence of MD3 together with its downstream sequence on the mRNA until the polyA tail.

TABLE 2
Description of domains in FIG. 5

| Domain | | Functionally equivalent |
|---|---|---|
| name | Domain function | domain in FIG. 1 |
| DA | Primer binding site, suitable | P2 |
| | for circularization | |
| CB | Compartment or cell barcode | Part of BC |
| UMI | Unique Molecular Identifier | Part of BC |
| PolyT | Reverse transcription primer | N/A, |
| | that binds poly A tail of mRNA | |
| PolyA | Originated from part of the | N/A |
| | poly A tail on the mRNA | |
| MU | A ~20 nt sequence on the | Part of sequence of |
| | mRNA upstream to the | interest, 5′ end of |
| | potential mutation site | MU marks the |
| | | truncation site 102 |
| DU | Primer binding site, | X |
| | capable of circularization | |
| MD5 | A ~20-nt sequence on the | Part of sequence of |
| | mRNA downstream to the | interest, 3′ end of |
| | potential mutation site | MD5 marks the |
| | | truncation site 105 |
| MD3 | A ~20-nt sequence close to | P3 |
| | the 3′ end of the mRNA, | |
| | primer binding site | |
| DB | Adaptor sequence, possibly | P4 |
| | sequencing primer-binding | |
| | site | |
| DC | Adaptor sequence | N/A |
| Rd1, Rd2 | Exemplary sequences of DA | N/A |
| | and DB* domains (as in | |
| | Illumina platform) | |
| P5, P7 | Exemplary domains necessary | N/A |
| | to perform NGS | |
| i5 (index | Sample indices | N/A |
| read2 in | | |
| Illumina | | |
| platform) | | |

FIGS. 6A to 6C show an improved version of the DropSeq-like method. In FIG. 6A, Step 6.1 illustrates the tagmentation of multiple copies of cDNA molecules (601) into truncated cDNA molecules (602, 603, and 604), of different lengths. In this process, additional domain DC*/DC are attached to the DNA break points. Note that in this improved version the RT primer is designed so that the cDNA molecules have an additional domain DB*/DB. The fragmented cDNA molecules are circularized to obtain circular DNA (605, 606, and 607) (Step 6.2). FIG. 6B shows the circular DNA being subject to another tagmentation reaction and the introduction of domain DD*/DD to obtain linear DNA molecules (651, 652 and 653). Primers DB* and DD can be used to amplify these linear DNA molecules to produce amplified linear DNA molecules (654, 655, 656). These amplified linear DNA molecules may be sequenced (dashed arrows on 657 show the regions that can be sequenced). Molecule 657 illustrates the original cDNA molecule (i.e., the same as 601, 602, and 603). FIG. 6C illustrates how new domains (e.g., P5, P7, i5) can be introduced to the amplified DNA molecules (658, which is a collective representation of 654, 655, and 656) to produce adaptor-containing DNA molecules (659) which can be sequenced by NGS. FIGS. 6A to 6C can be considered an example of FIG. 1. Table 3 discloses what each domain name in FIGS. 6A, 6B, and 6C represents.

TABLE 3
Description of domains in FIGs. 6A, 6B, and 6C

| Domain | | Functionally equivalent |
|---|---|---|
| name | Domain function | domain in FIG. 1 |
| DA | Primer binding site, suitable | P2 |
| | for circularization | |
| CB | Compartment or cell barcode | Part of BC |
| UMI | Unique Molecular Identifier | Part of BC |
| PolyT | Reverse transcription primer | N/A, |
| | that binds poly A tail of mRNA | |
| PolyA | Originated from part of the | N/A |
| | poly A tail on the mRNA | |
| DB | Primer binding site | P3 |
| DC | Adaptor sequence, suitable | X |
| | for circularization | |
| DD | Adaptor sequence | P4 |
| TA, TB, TC | Sequence of interest | Sequence of interest |
| TX | Collective reference to | Sequence of interest |
| | domains TA, TB, and TC | |
| Rd1, Rd2 | Exemplary sequences of DA | N/A |
| | and DD domains (as in | |
| | Illumina platform) | |
| P5, P7 | Exemplary domains necessary | N/A |
| | to perform NGS | |
| i5 (index | Sample indices | N/A |
| read2 in | | |
| Illumina | | |
| platform) | | |

FIG. 7 illustrates three distinct biological particles processed to obtain three pools of nucleic acid molecules containing target nucleic acid sequences, barcoding of the nucleic acid molecules with a barcode unique to the distinct biological particle from which the barcoded nucleic acid molecule originated, circularization of the barcoded nucleic acid molecules to obtain circular barcoded nucleic acid molecules, and linearizing the circular barcoded nucleic acid molecules to obtain truncated and barcoded nucleic acid molecules having a truncated portion of the target polynucleotide sequence.

FIG. 8 provides an example of BCR/TCR-transcriptome co-sequencing using a panel of primers for second strand synthesis (SSS). In Step 8.1, the TCR/BCR transcript is reverse-transcribed by the RT primer (801), which contains cell barcode ($CB) and molecular barcode ($UMI). In Step 8.2, the cDNA molecules are converted to amplified dsDNA molecules using a panel of SSS primers (803) and appropriate PCR primers. The SSS step also serves as a truncation step. In Step 8.3, circularization domains ($X/$X*) and optional sample indices are appended to the two ends of the amplified dsDNA molecules using PCR. The PCR product is circularized (Step 8.4). Then the circularized DNA molecules are linearized by PCR using primers 804 and 805. FIG. 8 can be considered an example of FIG. 1. Table 4 discloses what each domain name in FIG. 8 represents. Domains V, D, J, C have the same meaning as in FIG. 4. Domain Vt means truncated domain V. The exact sequences of some of these domains are shown in Table 5.

TABLE 4
Description of domains in FIG. 8

| Domain | | Functionally equivalent |
|---|---|---|
| name | Domain function | domain in FIG. 1 |
| $Rd1 | Primer binding site, | P2 |
| | suitable for (1) | |
| | amplification, (2) Illumina | |
| | sequencing and (3) with | |
| | modification, | |
| | circularization | |
| $CB | Compartment or cell barcode | Part of BC |
| $UMI | Unique Molecular Identifier | Part of BC |
| $PolyT | Reverse transcription primer | N/A, |
| | that binds poly A tail of | |
| | mRNA | |
| $C3 | Primer binding site close | P3 |
| | to the 3′ of the C segment | |
| | in TCR/BCR | |
| $X | Adaptor sequence, suitable | Part of X |
| | for circularization | |
| $Idx | Optional sample index | Part of X |
| $zRd2 | Illumina sequencing primer | Part of X |
| | binding site | |
| [$X* \| | Adaptor sequences with | X |
| $Idx* \| | multiple functions, | |
| $zRd2} | including circularization, | |
| | sample indexing, and | |
| | sequencing | |
| $P5 | Adaptor sequence, suitable | P4 |
| | for amplification and | |
| | Illumina sequencing | |
| $C5 | Part of sequence of | Part of sequence |
| | interest, primer binding | of interest. The |
| | site for truncation | 3′ end of $C5 |
| | | marks 105 |
| $zP7 | Adaptor sequence suitable | Sequence of |
| | for amplification and | interest |
| | Illumina sequencing | |

FIG. 9 shows the scheme to test the circularization efficiency using qPCR (see Example 1). Primer sequences are shown in Table 7. The sequences of TRA and TRB genes are shown in Table 8.

FIG. 10 shows the results of circularization efficiency test using qPCR (see Example 1).

### DESCRIPTION OF THE SEQUENCES

Domain level description. In this document, sometimes the polynucleotide sequence is described at domain level. Each domain name corresponds to a specific polynucleotide sequence and/or a specific function. For example, domain ‘A’ may have a sequence of 5′-TATTCCC-3′, domain ‘B’ may have a sequence of 5′-AGGGAC-3′, and domain ‘C’ may have a sequence of 5′-GGGAAGA-3′. In this case the polynucleotide having a sequence that is the concatenation of domains A, B, and C, can be written as [A|B|C}. The symbol ‘[’ denotes the 5′ end, the symbol ‘}’ denotes the 3′ end, and the symbol ‘|’ separates domain names. An asterisk sign shows sequence complementarity. For example domain ‘B*’ is the reverse complement of domain ‘B’.

Functional description vs sequence description. In some figures and descriptions in this document (e.g. FIG. 1), names of domains (e.g., P1, P2, and BC) describe the function of the domain. The exact sequence of these domains may vary depending on platform or application. In other figures and descriptions (e.g., FIG. 8 and associated text), some names of domains (e.g., Rd1 and zRd2 in FIG. 8) describe the specific sequence of the domain. To distinguish these two types of annotation, we add a dollar sign ($) to the front of the names of domains that describe specific sequences. In other words, each domain name with a $ sign in this document is associated with a specific sequence. Some of these domain names are listed in Table 5. Note that ‘specific sequence’ may a fixed or variable sequence. For example ‘$UMI’ is a random hexamer and may be any hexamer sequence, ‘$CB’ is the cell barcode used in Klein et al., which contains two variable barcode regions.

Table 5 provides a listing of certain sequences referenced herein.

TABLE 5
Description of the Sequences

| | | SEQ |
|---|---|---|
| | | ID |
| Description | Sequence | NO |
| $Rd1 | 5′-ACACTCTTTCCCTACACGACGCTCTTCCGATCT-3′ | 1 |
| $CB | 5′-XXXXXXGAGTGATTGCTTGTGACGCCTXXXXXX-3′ | 2 |
| $UMI | 5′-NNNNNN-3′ | 3 |
| $PolyT | 5′-TTTTTTTTTT TTTTTTTTTT C-3′ | 4 |
| $zRd2 | 5′-GTGACTGGAGTTCAGACGTGTGCTCTTCCGATC-3′ | 5 |
| $zRd1^{Δ} | 5′-ACACTCTTTCCCTACACGAC-3′ | 6 |
| $zRd2^{Δ} | 5′- GTGACTGGAGTTCAGACGTGT-3′ | 7 |
| $X | 5′- GGCGGGCGCG-3′ | 8 |
| $P5 | 5′-AATGATACGGCGACCACCGAGA-3′ | 9 |
| $C5 | 5′-CCGTGTACCAGCTGAGAGACT-3′fom | 10 |
| $zP7 | 5′-CAAGCAGAAGACGGCATACGAGAT-3′ | 11 |
| $C3 | 5′-GGATCTTCAGTGGGTTCTCTTG-3′ | 12 |
| $P01 | 5′- | 13 |
| | /Phos/CGCGCCCGCCATACTCTTTCCCTACACGACGCTCT | |
| | -3′ | |
| $P02 | 5′- | 14 |
| | GGCGGGCGCGATTTCGCCTTAGTGACTGGAGTTCAGA | |
| | CGTG-3′ | |
| $P03 | 5′-CGTCAGGTGGAAGGAGGTTTC-3′ | 15 |
| $P04 | 5′-GGCGTGTTGTATGTCCTGCTG-3′ | 16 |
| $P05 | 5′-CTGAGGGCTGGATCTTCAGAGTG-3′ | 17 |
| $P06 | 5′-GGACCTTAGCATGCCTAAGTGAC-3′ | 18 |
| $P07 | 5′-TCAAGCTGGTCGAGAAAAGCT-3′ | 19 |
| $P08 | 5′-ATTAAACCCGGCCACTTTCAG-3′ | 20 |
| $TRA | 5′- GTGACTGGAGTTCAGACGTGTGCTCTTCCGATCT | 21 |
| | AGAGTGAAACCTCCTTCCACCTGACGAAACCCTCAGCCC | |
| | ATATGAGCGACGCGGCTGAGTACTTCTGTGCTGTGAGT | |
| | GANNGGGGTACAGCAGTGCTTCCAAGATAATCTTTGG | |
| | ATCAGGGACCAGACTCAGCATCCGGCCAANATATCCAG | |
| | AACCCTGACCCTGCCGTGTACCAGCTGAGAGACTCTAA | |
| | ATCCAGTGACAAGTCTGTCTGCCTATTCACCGATTTTGA | |
| | TTCTCAAACAAATGTGTCACAAAGTAAGGATTCTGATGT | |
| | GTATATCACAGACAAAACTGTGCTAGACATGAGGTCTA | |
| | TGGACTTCAAGAGCAACAGTGCTGTGGCCTGGAGCAAC | |
| | AAATCTGACTTTGCATGTGCAAACGCCTTCAACAACAGC | |
| | ATTATTCCAGAAGACACCTTCTTCCCCAGCCCAGAAAGT | |
| | TCCTGTGATGTCAAGCTGGTCGAGAAAAGCTTTGAAAC | |
| | AGATACGAACCTAAACTTTCAAAACCTGTCAGTGATTG | |
| | GGTTCCGAATCCTCCTCCTGAAAGTGGCCGGGTTTAAT | |
| | CTGCTCATGACGCTGCGGCTGTGGTCCAGCTGAGATCT | |
| | GCAAGATTGTAAGACAGCCTGTGCTCCCTCGCTCCTTCC | |
| | TCTGCATTGCCCCTCTTCTCCCTCTCCAAACAGAGGGAA | |
| | CTCTCCTACCCCCAAGGAGGTGAAAGCTGCTACCACCTC | |
| | TGTGCCCCCCCGGTAATGCCACCAACTGGATCCTACCCG | |
| | AATTTATGATTAAGATTGCTGAAGAGCTGCCAAACACT | |
| | GCTGCCACCCCCTCTGTTCCCTTATTGCTGCTTGTCACT | |
| | GCCTGACATTCACGGCAGAGGCAAGGCTGCTGCAGCCT | |
| | CCCCTGGCTGTGCACATTCCCTCCTGCTCCCCAGAGACT | |
| | GCCTCCGCCATCCCACAGATGATGGATCTTCAGTGGGT | |
| | TCTCTTGGGCTCTAGGTCCTGGAGAATGTTGTGAGGG | |
| | GTTTATTTTTTTTTAATAGTGTTCATAAAGAAATACATA | |
| | GTATTCTTCTTCTCAAGACGTGGGGGGAAATTATCTCAT | |
| | TATCGAGGCCCTGCTATGCTGTGTGTCTGGGCGTGTTG | |
| | TATGTCCTGCTGCCGATGCCTTCATTAAAATGATTTGGA | |
| | AAAAAAAAAAAAAAAAAAAGATCGGAAGAGCGTCGTG | |
| | TAGGGAAAG-3′ | |
| $TRB | 5′- GTGACTGGAGTTCAGACGTGTGCTCTTCCGATCT | 22 |
| | CCACTCTGAAGATCCAGCCCTCAGAACCCAGGGACTCA | |
| | GCTGTGTACTTCTGTGCCAGCAGTTTAGCNGGGACAGG | |
| | GGGCNCTAACTATGGCTACACCTTCGGTTCGGGGACCA | |
| | GGTTAACCGTTGTAGNAGGACCTGAACAAGGTGTTCCC | |
| | ACCCGAGGTCGCTGTGTTTGAGCCATCAGAAGCAGAGA | |
| | TCTCCCACACCCAAAAGGCCACACTGGTGTGCCTGGCC | |
| | ACAGGCTTCTTCCCTGACCACGTGGAGCTGAGCTGGTG | |
| | GGTGAATGGGAAGGAGGTGCACAGTGGGGTCAGCAC | |
| | GGACCCGCAGCCCCTCAAGGAGCAGCCCGCCCTCAATG | |
| | ACTCCAGATACTGCCTGAGCAGCCGCCTGAGGGTCTCG | |
| | GCCACCTTCTGGCAGAACCCCCGCAACCACTTCCGCTGT | |
| | CAAGTCCAGTTCTACGGGCTCTCGGAGAATGACGAGTG | |
| | GACCCAGGATAGGGCCAAACCCGTCACCCAGATCGTCA | |
| | GCGCCGAGGCCTGGGGTAGAGCAGACTGTGGCTTTAC | |
| | CTCGGTGTCCTACCAGCAAGGGGTCCTGTCTGCCACCA | |
| | TCCTCTATGAGATCCTGCTAGGGAAGGCCACCCTGTAT | |
| | GCTGTGCTGGTCAGCGCCCTTGTGTTGATGGCCATGGT | |
| | CAAGAGAAAGGATTTCTGAAGGCAGCCCTGGAAGTGG | |
| | AGTTAGGAGCTTCTAACCCGTCATGGTTTCAATACACAT | |
| | TCTTCTTTTGCCAGCGCTTCTGAAGAGCTGCTCTCACCT | |
| | CTCTGCATCCCAATAGATATCCCCCTATGTGCATGCACA | |
| | CCTGCACACTCACGGCTGAAATCTCCCTAACCCAGGGG | |
| | GACCTTAGCATGCCTAAGTGACTAAACCAATAAAAATGT | |
| | TCTGGTCTGGCCTGAAAAAAAAAAAAAAAAAAAAGATC | |
| | GGAAGAGCGTCGTGTAGGGAAAG-3′ | |

### DETAILED DESCRIPTION

### I. Definitions

Biological particles: “Biological particles” are individually separable and dispersible particles of biological origin, such as cells (prokaryotic or eukaryotic), nuclei, cell clusters, organelles (such as mitochondria), and viruses. Other than viruses, biological particles are usually composed of at least 50 molecules and are usually large enough that they cannot pass through 0.22-micron filter. In some embodiments, the biological particles are prepared from biological samples. For example, the biological particles can be cells prepared from fresh tissue (such as dense cell matter from tumor or neural tissues). In some embodiments, the biological particles are whole cells or nuclei prepared from frozen tissue. See Krishnaswami et al., Nat. Protoc. 11:499-524 (2016). In some situations, the analysis of nuclei (rather than cells) may be advantages or necessary. For example, when the cells are abnormally shaped cells (e.g. neurons) or when freezing conditions have ruptured the outer cell membrane, intact cells can be difficult to prepare, whereas intact nuclei can be prepared more readily.

In some embodiments, at least some of the cells can be engineered with DNA, RNA, or viral vectors that encode one or more biological agents that cause RNA-mediated gene knockdown, genome editing, transcriptional alteration, or epigenetic alteration. The one or more biological agents may include, for example, one or more of siRNA, shRNA, miRNA, zinc finger domains, transcription activator-like effector (TALE), Cas9, or RNA with CRISPR origin.

Cell Clusters: As used herein, “cell clusters” refer to a grouping of cells. In some embodiments, the cell clusters comprise cells that express an antigen-recognizing agent and cells that express an antigen. Antigen-recognizing agents include, for example, an antigen-recognizing protein, such as an antibody, functional antibody fragment, or a T-cell receptor (TCR), or an antigen-recognizing polynucleotide. In some embodiments, the cell cluster comprises T cells and antigen presenting cells (APCs). The antigen may be complexed, for example, with a major histocompatibility complex (MHC) molecule.

Barcode: As used herein, a “barcode” or “BC” refers to a sequence barcode or barcodes responsible for deciphering the original location, count, or identity of the nucleic acid molecule. In some embodiments, the barcode comprises a compartment barcode (CB) and/or a unique molecular identification (UMI) sequence. To accomplish the barcoding, it is only necessary to bind a single barcode to the nucleic acid molecule. The length of a barcode may be from 3 to 20 nucleotides, 4 to 10 nucleotides, or 6 to 8 nucleotides in length, or 3, 4, 5, 6, 7, 8, 9, 10, 15, or 20 nucleotides in length.

Compartment barcode: A “compartment barcode” or “CB” is a nucleic acid sequence that is carried by primers that denote the identity of the compartment a target nucleic acid was associated with. Compartment barcode usually varies between compartments (i.e., different compartments have different compartment barcodes). At the same time, all compartment barcode sequences on all primers in one compartment usually are, or are intended to be, the same. The length of a barcode may be from 3 to 20 nucleotides, 4 to 10 nucleotides, or 6 to 8 nucleotides in length, or 3, 4, 5, 6, 7, 8, 9, 10, 15, or 20 nucleotides in length.

The compartment barcode is often created by clonal expansion of single template nucleic acid molecules (e.g., Church and Vigneault, US20130274117) or by split-and-pool synthesis (e.g., in inDrop™ and DropSeg™ technologies, see Klein et al. above and Macosko et al., Cell 161:1202-1214 (2015), respectively).

In some embodiments, a compartment barcode is a cell barcode. See, e.g., Klein et al. above. For example, in single cell RNA-Seq techniques, such as Drop-Seg™ and inDrop™, compartment barcodes are used as cell barcodes, such that all RNA transcripts from the same cell are reverse-transcribed off primers sharing the same compartment barcode.

Unique molecular identification (UMI) sequence: As used herein, a “unique molecular identification” or “UMI” sequence refers to short oligonucleotides added to each molecule in some NGS protocols prior to amplification. The UMI may include random nucleotides (e.g., NNNNNNN), partially degenerate nucleotides (e.g., NNNRNYN), or defined nucleotides (e.g., when template molecules are limited). The use of UMIs can reduce the quantitative bias introduced by replication, which may be necessary to have enough molecules for detection, as duplicate molecules may be identified. In some embodiments, the length of an UMI is from 3 to 10 or 4 to 8 bp in length, or 3, 4, 5, 6, 7, 8, 9, or 10 bp in length.

Primer: Primers are oligonucleotides that, during an experiment or a series of experiments, become part of a molecule or a molecular complex comprising: (a) the primer; and (b) a nucleic acid moiety that is either a target nucleic acid or a nucleic acid moiety whose formation is dependent on the presence or sequence of the target nucleic acid. As used herein, “primer” includes a single primer or a panel of different primers. In some embodiments, one or more of the primers may have an extendable 3′ end, may hybridize to a template nucleic acid (DNA or RNA), and/or may be extended by polymerases to copy the template nucleic acid (such as the target nucleotide sequence). In some embodiments, one or more of the primers may be a substrate for ligation. In some embodiments, one or more of the primers may participate in a hybridization or crosslinking reaction.

One or more of the primers may be engineered or chosen based on the features of target nucleotide sequence. The primers usually have at least 4, 5, or 6 consecutive nucleotides that are complementary to at least a portion of the target nucleotide sequence. One or more of the primers may comprise a non-specific sequence (e.g., oligo/poly (d)T/U) or gene-specific sequence. As an example, if the target nucleic acid is polyadenylated RNA, oligo dT primer can be used as primer. The oligo dT primer anneals to the polyA tail of the RNA. In other embodiments, a gene-specific primer can be used. Gene-specific primers are designed based on known sequences of the target RNA. Gene-specific primers are commonly used in one-step RT-PCR applications.

The length of one or more of the primers may be from 4 to 200,80 to 160, or 120 to 140 nucleotides in length, or 4, 5, 6, 8, 10, 20, 30, 40, 50, 60, 70, 80, 90, 100, 110, 120, 130, 140, 150, 160, 170, 180, 190, or 200 nucleotides in length. In some embodiments, the primer is also associated with a unique molecular identification (UMI) sequence and/or a barcode (BC) sequence. Methods to design primers to known sequence are well known to a person of ordinary skill in the art.

In some embodiments, one or more of the primers may contain randomly synthesized sequence, alone or in combination with an oligo dT primer. Randomly synthesis gives a range of sequences with potential to anneal at random points on a DNA sequence and act as a primer to start first strand cDNA synthesis in various PCR applications. In some embodiments, the randomly synthesized sequence is from 2 to 20, 3 to 15, or 4 to 10 nucleotides in length, or 2, 3, 4, 5, 6, 7, 8, 9, 10, 12, 14, 16, 18, or 20 nucleotides in length. For example, random hexamer or random hexonucleotides are commonly used when the sequence of target nucleotide sequence is unknown or diverse. See, e.g., Hansen et al., Nucleic Acids Res. 38:e131 (2010).

Primer delivery particle: As used herein, “primer delivery particle” refers to a particle that can host primers within, on the surface, or throughout the material comprising the particle. In some embodiments, the primer delivery particle also hosts a unique molecular identification (UMI) sequence and/or a barcode (BC) sequence and these sequences can be directly linked to the primer sequence. The primers may be attached to the primer delivery particle by methods known to those of skill in the art, such as by amine-thiol crosslinking, maleimide crosslinking, or crosslinking usingN-hydroxysuccinimide or N-hydroxysulfosuccinimide In some embodiments, biotin may be used to attach the primer to one or more beads coated with streptavadin.

In some embodiments, the diameter of a primer delivery particle can be about from 1 micron to 1 millimeter, or greater than or equal to 1, 5, 10, 30, 50, 100, 500, or 750 microns. The primer delivery particle can be of uniform or heterogeneous volume. The average volume of a batch of primer delivery particles used in one experiment may be from 0.5 femtoLiter to 0.5 microLiter, from 1.0 femtoLiter to 0.25 microLiter, or from 10 femtoLiter to 0.125 microLiter, or from 1 picoLiter to 5 nanoLiter.

In some embodiments, the primer delivery particle may be a droplet or fluid, such as a water in oil droplet or lipid microsphere that contains the primers internally in an aqueous solution. A primer delivery particle may also be a “solid,” such as a bead, or a soft, compressible, yet non-fluidic material, such as a hydrogel (e.g., agarose gel, polyacrylamide gel, and polydimethylsiloxane (PDMS) gel, such as polyethylene glycol (PEG)/PDMS hydrogel).

A bead may encompass any type of solid or hollow sphere, ball, bearing, cylinder, or other similar configuration composed of plastic, ceramic, metal, or polymeric material onto which a nucleic acid may be immobilized (e.g., covalently or non-covalently). A bead may comprise nylon string or strings. A bead may be spherical or non-spherical in shape. Beads may be unpolished or, if polished, the polished bead may be roughened before treating (e.g., with an alkylating agent). A bead may comprise a discrete particle that may be spherical (e.g., microspheres) or have an irregular shape. The diameter of the beads may be about 5 μm, 10 μm, 20 μm, 25 μm, 30 μm, 35 μm, 40 μm, 45 μm, 50 μm, 60 μm, 70 μm, 80 μm, 90 μm, or 100 μm. A bead may refer to any three-dimensional structure that may provide an increased surface area for immobilization of biological particles and macromolecules, such as DNA and RNA. Beads may comprise a variety of materials including, but not limited to, paramagnetic materials, ceramic, plastic, glass, polystyrene, methylstyrene, acrylic polymers, titanium, latex, sepharose, cellulose, nylon, agarose, polyacrylamide, and the like. Examples of beads include the gel bead GEMs in Zheng et al., Nat. Commun. 8:14049 (2017) and the gel beads in Klein et al.

The terms “hydrogel”, “gel,” and the like, are used interchangeably herein and may refer to a material which is not a readily flowable liquid and not a solid but a gel of from 0.25% to 50%, 0.5% to 40%, 1% to 30%, or 5% to 25%, or 0.5%, 1%, 5%, 10%, 20%, 30%, 40%, or 50%, by weight of gel forming solute material, and from 45% to 98%, 55% to 95%, 60% to 90%, or 65% to 85% by weight of water. The gels may be formed, for example, using a solute, synthetic or natural (e.g., for forming gelatin) to form interconnected cells which bind, entrap, absorb and/or otherwise hold water to create a gel, which may include bound and unbound water. The gel may be a polymer gel.

Primer binding site: As used herein, a primer binding site is a region of a nucleotide sequence where a RNA or DNA single-stranded primer binds to start replication.

Target polynucleotide sequence: A target polynucleotide sequence is the polynucleotide sequence selected for analysis, wherein the analysis can be any procedure that produces a human- or computer-observable signal. The analysis may comprise polymerase chain reaction (PCR), quantitative PCR (qPCR), Sanger sequencing, or NextGen sequencing (NGS, using platforms such as Illumina MiSeg™, Illumina HiSeg™, Illumina NextSeg™ Illumina NovaSeg™, Ion Torrent, SOLiD™, Roche 454, and the like), and the like. The analysis may yield information about the sequence or quantity of the target polynucleotide sequence. A target polynucleotide sequence can be DNA, RNA, or modified nucleic acid, such as bisulfite-treated DNA. The target polynucleotide sequence is at least part of an engineered molecule that is used to engineer or probe the biological particle. Thus, the target polynucleotide sequence may be the entirety or a subset of the genome or the transcriptome. The target polynucleotide sequence may be endogenous to the biological particle it resides in (i.e., it is in the biological particle without human intervention), or be exogenous to the biological particle it resides in (i.e., it is in the biological particle due entirely or partly to human intervention). The target polynucleotide sequence may be exogenously expressed mRNA, shRNA, non-coding RNA, or guide RNA (for the CRISPR/Cas9-based system). The target polynucleotide sequence may contain a barcode sequence. In some embodiments, the target polynucleotide sequence comprises one or more of a partial or complete T cell or B cell receptor sequence, a mutation, a transcription start site, or a splicing junction.

The target polynucleotide sequence may be a synthetic nucleic acid molecule that is conjugated to a detection probe, such as monoclonal antibody. Sometimes the original target nucleic acid one intends to analyze is converted to another molecular species or molecular complex such as a hybridization product, a primer-extension product (where the original target nucleic acid acts as the template or primer), a PCR product (where the original target nucleic acid acts as the template), a ligation product (where the original target nucleic acid acts as the splint, the 5′ ligation substrate or the 3′ ligation substrate). The newly created molecular species or molecular complexes can also be considered target polynucleotide sequence.

Template-Switching Oligonucleotide: As used herein, a “template-switching oligonucleotide” (TS oligo or TSO) refers to a DNA oligo sequence primer that carries additional consecutive bases at the 3′ end (e.g., 3 riboguanosines (rGrGrG)). The complementarity between these consecutive bases and the 3′ extension of the cDNA molecule empowers the subsequent template switching. Turchinovich et al., RNA Biol. 11(7):817-828 (2014). The sequence of the TSO (other than the consecutive Gs at the 3′ end) is largely arbitrary. The length of a TSO is equal to or greater than 3, 4, 5, 10, 20, or 30 nucleotides in length. In some embodiments the TSO is from 15 to 30 nucleotides in length.

A TSO may be used, for example, in methods such as template-switching polymerase chain reaction (TS-PCR) to produce cDNA from RNA. Petalidis et al., Nucleic Acids Res. 31(22):e142 (2003). TS-PCR is a method of reverse transcription and polymerase chain reaction (PCR) amplification that relies on a natural PCR primer sequence at the polyadenylation site and adds a second primer through the activity of murine leukemia virus (MLV) reverse transcriptase. Examples of TS-PCR include the SMART™ (switching mechanism at the 5′ end of the RNA transcript) or SMARTer™ methods of Clontech Laboratories, and the CATS™ (capture and amplification by tailing and switching) of Diagenode Inc.

In one example, upon reaching the 5′ end of the RNA template during first-strand synthesis, the terminal transferase activity of the MLV (e.g., Moloney murine leukemia virus or MMLV) reverse transcriptase adds a few additional nucleotides (mostly deoxycytidine) to the 3′ end of the newly synthesized cDNA strand. These bases function as a TSO-anchoring site. Upon base pairing between the TSO and the appended deoxycytidine stretch, the reverse transcriptase “switches” template strands, from cellular RNA to the TSO, and continues replication to the 5′ end of the TSO. The resulting cDNA contains the complete 5′ end of the transcript, and universal sequences of choice can be added to the reverse transcription product. Along with tagging of the cDNA 3′ end by oligo dT primers, one may amplify the entire full-length transcript pool in a sequence-independent manner. Shapiro et al., Nat. Rev. Genet. 14(9):618-630 (2013).

Circularizing: As used herein, “circularizing” refers to the conversion of a linear nucleic acid molecules into a circular form. Circularization may be obtained by, for example, homologous recombination of the ends or by association of complementary single stranded ends (sticky ends). Circularization may also be obtained by ligating the two ends of the linear nucleic acids. The ligation can be blunt-end ligation or sticky-end ligation. In some embodiments, the length of circular barcoded nucleic acid molecules is equal to or greater than 1 kb, 1.5 kb, 2 kb, 3 kb, 5 kb, or 10 kb.

Linearizing: As used herein, linearizing refers the conversion of circular nucleic acid molecules to a linear form by fragmentation. Linearization may be accomplished by physical (e.g., acoustic, sonication, hydrodynamic), enzymatic (e.g., transposase, DNase I or other restriction endonuclease, non-specific nuclease), and/or chemical (e.g., heat and divalent metal cation, such as magnesium or zinc) methods. In some embodiments, linearization is by enzymatic means, such as through use of a transposase.

Tagmentation. As used herein, tagmentation refers to fragmentation and tagging of double-stranded DNA using a transposase, such as Tn5 transposase (e.g., Nextera™ methods by Illumina).

### II. Overall Process

A typical barcoded nucleic acid molecule has the structure shown in FIG. 1A, where P1 and P2 are primer binding site, BC is the barcode, and the thin line represents the full sequence of interest which that can be very long (e.g., of varying length and sometimes >1 kb). Using prior methods only the region in the sequence of interest close to the BC (e.g., within approximately 500 bp (base pairs)) can be sequenced. To obtain sequence distant from the BC (such as a sequence greater than 500 bp, greater than 750 bp, greater than 1000 bp, etc. from the BC), one can use the following strategy.

Step 0. Ensure there is a functional primer-binding site between the BC and the sequence of interest. An additional primer binding site P3 between BC and the sequence of interest can be strategically added, for example, during primer synthesis (e.g., by including P3 sequence in the primer extension template during the split-and-pool primer synthesis for inDrop™ technology). Poly A and poly T sequence may also serve as P3. As a result, the barcoded long DNA molecule has the structure shown in 101 of FIG. 1B.

Step 1 (optional). Create a truncated molecule that optionally includes an additional domain X. FIG. 1B shows the site of truncation (102). The truncated molecule can be created by multiple methods, including but not limited to: (a) cleaving the molecule (101) mechanically or enzymatically; (b) using a Tn5 transposase which may be complexed with an oligonucleotide adaptor; or (c) extending off a primer that recognizes the sequence near the truncation site. The primer can be of a defined sequence or of a random sequence. For example, if one is interested in a specific region of DNA such as the region around a possible point mutation or hypervariable region (e.g., B-Cell Receptor (BCR) or T-Cell Receptor (TCR) sequence), one may use a primer of a defined sequence. Alternatively, if one is interested in surveying the transcriptome in an unbiased fashion, one may use a primer of a random sequence (e.g., a random hexamer). If needed, the domain X can be added by methods that include, but are not limited to, ligating it to the cleavage site (if method (a) above is used), including it in the oligonucleotide adaptor that is complexed with the Tn5 transposase (if method (b) above is used), or by including it at the 5′ end of the primer (if method (c) above is used). In some embodiments, the optional domain X may be useful during the circularization step below (Step 2).

Step 2. Circularize the truncated molecule (103) to join the free end of P2 and the other end of the truncated molecule (optionally with domain X in between) to form a circular DNA (104) of FIG. 1B. The truncated molecule that undergoes circularization can be in the form of single-stranded DNA (ssDNA) or double-stranded DNA (dsDNA). A truncated molecule in ssDNA form can be obtained from dsDNA form by, for example, heating. ssDNA can then be circularized, for example, by CircLigase™ ssDNA ligase from Epicentre Biotechnologies. In some embodiments, a “splint” or “bridge” oligonucleotide that interacts with the two termini can be used to facilitate the circularization of ssDNA, in which case a more traditional DNA ligase, such as T4 DNA ligase, may be used. A domain X can facilitate the design of such a splint because the sequence of domain X is often known.

If the truncated molecule is in dsDNA form, the ligation can be made between blunt ends or sticky ends. The sticky end can be created by multiple mechanisms, such as: (a) cleavage with a restriction enzyme; (b) embedding a deoxyuridine base followed by cleavage with USER™ enzyme mix (New England BioLabs, see, e.g., Geu-Flores et al., Nucleic Acids Res. 35(7):e55 (2007)); (c) using a 5′-to-3′ exonuclease activity as in the Gibson Assembly (Gibson et al., Nat. Methods 6:343-345 (2009)); or (d) using 3′-to-5′ exonuclease activity as in ligation-independent cloning (LIC) (Aslandidis et al., Nucleic Acids Res. 18:6069-74 (1990)) or sequence and ligation-independent cloning (SLIC) (Li et al., Nat. Methods 4:251-256 (2007)).

Promotion of intra-molecular circularization and minimization of inter-molecular ligation may be achieved by: (a) compartmentalizing the molecules in a large number (e.g., millions or more) of small compartments (e.g., droplets); (b) adding reagents that reduce diffusion (e.g., glycerol); or (c) immobilizing the DNA on a surface or to polymer in a hydrogel to restrict free diffusion. If the substrate is ssDNA, an oligo complementary to a constant region on the substrate (e.g., P3) can be used to immobilize the substrate DNA molecule on a solid surface or to a polymer. If the substrate is dsDNA, a dsDNA-binding protein, such as a catalytically inactive form of a restriction enzyme, Zinc-Finger Protein, TALE protein, and dCas9/gRNA complex, can be used to immobilize the substrate DNA on the solid surface or to a polymer. Immobilization can also be achieved, for example, by attaching a biotin moiety to the DNA and attaching the DNA to a surface or a polymer modified with streptavidin, or by covalently attaching DNA to a surface or a polymer. Optionally, linear (i.e., non-circularized) DNA can be removed by exonuclease treatment.

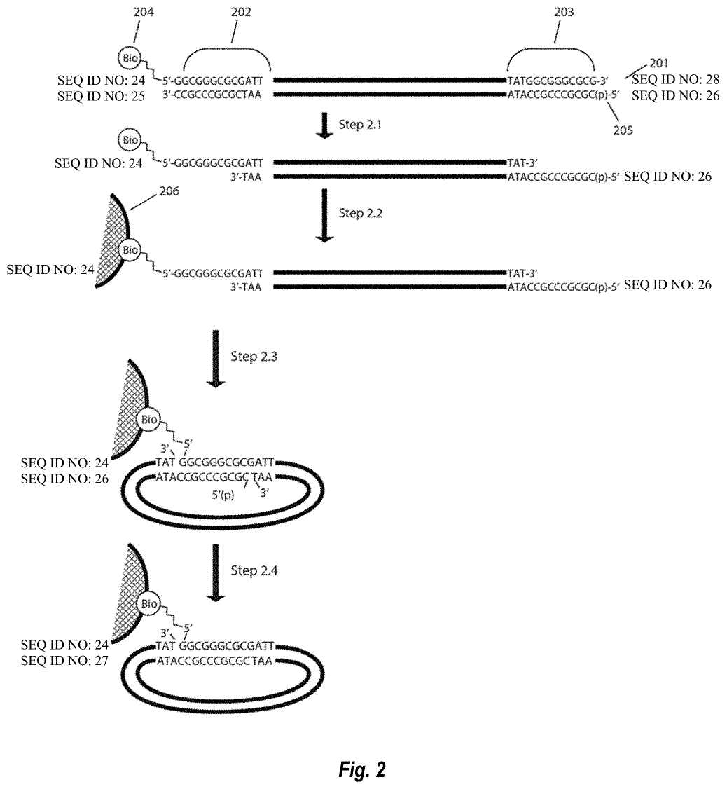

FIG. 2 illustrates an exemplary circularization method. The linear dsDNA is shown in black thick lines. First, the linear dsDNA is appended with additional double-stranded domains (202) and (203) on each end to form a modified linear dsDNA (201). Note that (202) and (203) share an identical stretch of sequence (i.e., 5′-GGCGGGCGCG-3′ on the top strand) to facilitate circularization. The 5′ end of top strand may also be modified with biotin (204) via a flexible linker. The length of the linker can be modified and optimized using methods known to skilled artisans. The 5′ end of the bottom strand is modified with a phosphate group (205). In Step 2.1 of FIG. 2, the 3′ end of each strand is degraded with an enzyme having 3′-to-5′ exonuclease activity to form unpaired, ‘sticky’ 5′ ends. The length of the degradation can be precisely controlled.

In this example, the additional domains (202) and (203) are designed in the way that the 3′ of each strand contain a stretch of sequence containing strictly A and T (e.g., 5′-TAT-3′ on the top strand and 5′-AAT-3′ on the bottom strand), followed by a stretch of sequence containing strictly G and C (e.g., 5′-GGCGGGCGCG-3′ on the top strand and 5′-CGCGCCCGCC-3′ on the bottom strand). The dsDNA can be treated, for example, with a DNA polymerase with 3′-to-5′ exonuclease activity and/or proof-reading activity (e.g., KOD (Thermococcus kodakaraenis) and Pfu (Pyrococcus furiosus) DNA polymerases) in the presence of dATP (deoxyadenosine triphosphate) and dTTP (deoxythymidine triphosphate), but not dCTP (deoxycytidine triphosphate) or dGTP (deoxyguanosine triphosphate). This way the DNA polymerase will keep degrading the G and C nucleotides on the 3′ of the DNA until it meets the A or T on the template where it will go back and forth between degrading the nucleotide and filling it back, likely favoring the latter. Other DNA polymerases that may be used include, but are not limited to, T7 DNA polymerase, DNA polymerase I, Taq DNA polymerase.

After creating the 5′ sticky end, the dsDNA can be immobilized on a solid surface. In some embodiments, the solid surface may be modified with streptavidin (206), such as streptavidin-coated magnetic beads, at low enough density that two dsDNA molecules are unlikely to reach each other. The condition used to immobilize the DNA on the surface should be such that hybridization of sticky ends is unfavorable. These conditions help to reduce or prevent inter-molecular ligation. In some embodiments, the order of Step 2.1 and Step 2.2 of FIG. 2 can be reversed. Namely, the linear dsDNA can be immobilized to a surface and then have the 3′ ends degraded.

Next, in Step 2.3 of FIG. 2, the immobilized linear DNA is circularized via hybridization between the two sticky ends on the 5′ ends. Then, in Step 2.4 of FIG. 2, the inner strand (originally bottom strand on the linear dsDNA) can be ligated using a DNA ligase, such as T4 DNA Ligase. In some embodiments, only one strand is circularized. The shared sequence in domains 202 and 203 (i.e., 5′-GGCGGGCGCG-3′ on the top strand) is an example of the optional domain X shown in FIGS. 1 and 3.

Step 3. Truncate the circularized molecule to form truncated linear molecule (106), while introducing a new primer-binding site P4 within proximity (e.g., less than or equal to 1000 bp, 900 bp, 800 bp, 700 bp, 600 bp, 500 bp, 400 bp, 300 bp, 200 bp, 100 bp, or 500 bp) of P3. Position 105 of FIG. 1B shows the site at which the new primer-binding site (P4) is added (i.e., the truncation site). The primer-binding site (P4) may be added, for example, using a method similar to the method described in Step 1, except that domain P4 replaces domain X. For example, Tn5 transposase complexed with P4-containing oligonucleotides can be used to cleave the substrate DNA and add P4 to the newly cleaved end. Alternatively, a primer with P4 appended on its 5′ end can be used to copy the circular DNA (104). Again, depending on application, the primer can be of defined sequence or random sequence. As a result, a short (e.g., less than or equal to 1000 bp, 900 bp, 800 bp, 700 bp, 600, bp, 500 bp, 400 bp, 300 bp, 200 bp, 100 bp, or 50 bp) DNA segment that: (a) comprises both a barcode and a portion of sequence of interest originally distal to the barcode (e.g., >500 bp, >750 bp, >1,000 bp, >1,500 bp away, etc.); and (b) are flanked by two primer binding sites (i.e., P3 and P4) is created. An example of this short DNA segment is the DNA segment from the end of P3 to the beginning of P4 in (106) of FIG.

1B.

Step 4. Amplify the resulting truncated barcoded DNA segment using primers capable of binding to the primer binding sites (e.g., that recognize P3 and P4 of FIG. 1B) to form an amplification product (see (107) of FIG. 1B). In some embodiments, the 5′ of these primers can contain additional sequences that facilitate NGS, such as one or more of P5, P7, Rd1, Rd2, or index sequences (e.g., i5 and i7). Amplification may be accomplished by methods well known to a person of ordinary skill in the art, such as PCR (polymerase chain reaction). In some embodiments, the amplification product has a length of equal to or less than 1000, 900, 800, 700, 600, 500, 400, 300, 200, 100, 50, or 25 base pairs. The sequencing can be initiated from the P4 adaptor (depicted by 112) or from the X adaptor.

In some embodiments, the creation of the truncated molecule described in Step 1 can be omitted. This method can be used, for example, to study the sequence immediately adjacent to P1 (such as transcription start site). This method is illustrated in FIG. 3. In some embodiments, P1 and P2 can be directly linked, optionally via an additional domain X.

In some embodiments, the barcoded amplification product is sequenced by methods known to a person of ordinary skill in the art. For example, the barcoded amplification product may be sequenced by methods that include, but are not limited to, polymerase chain reaction (PCR), quantitative PCR (qPCR), Sanger sequencing, NextGen sequencing (NGS, using platforms such as Illumina MiSeg™, Illumina HiSeg™, Illumina NextSeg™, Illumina NovaSeg™, Ion Torrent, SOLiD™, Roche 454, and the like), and the like.

### III. Applications

The methods described herein can be used to achieve several functionalities in the context of scRNA-seq (single cell RNA sequencing), such as: (1) pairing a T-cell receptor (TCR) sequence with a 3′ expression profile of single cells; (2) pairing point mutation distal to 3′ end and 3′ expression profile of single cells; and (3) quasi full-length scRNA-seq. Rationale and methods for these applications are given in the Examples.

scRNA-seq measures the distribution of expression levels for each gene across a population of cells. scRNA-seq may be accomplished using methods known to those of skill in the art and variations thereof, such as SMART-seq™, Smart-seq2™, SMARTer™, CEL-seq™, CEL-seq2™, InDrop-seg™, Drop-seq™, MARS-seq™, SCRB-seg™, Seq-well™, STRT-seq™, etc. In some embodiments, scRNA-seq uses the SMARTer™ (Switching Mechanism At 5′ End of RNA Transcript) method.

The “T-cell receptor” or “TCR” as used herein is a molecule found on the surface of T cells, or T lymphocytes, that is responsible for recognizing fragments of antigen as peptides bound to major histocompatibility complex (MHC) molecules. The binding between TCR and antigen peptides is of relatively low affinity and is degenerate: that is, many TCRs recognize the same antigen peptide and many antigen peptides are recognized by the same TCR. Sewell, A. K., Nat. Rev. Imm. 12(9): 669-677 (2012). When the TCR engages with antigenic peptide and MHC (peptide/MHC), the T lymphocyte is activated through signal transduction, that is, a series of biochemical events mediated by associated enzymes, co-receptors, specialized adaptor molecules, and activated or released transcription factors.

The TCR is a disulfide-linked membrane-anchored heterodimeric protein generally consisting of highly variable alpha (α) and beta (β) chains. Janeway et al., Immunobiology: The Immune System in Health and Disease. 5th ed. Glossary: Garland Science (2001). Each chain is composed of two extracellular domains: a variable (V) region and a constant (C) region. The C region is proximal to the cell membrane, followed by a transmembrane region and a short cytoplasmic tail, while the V region binds to the peptide/MHC complex.

The V domain of both the TCR α-chain and β-chain each have three hypervariable or complementarity determining regions (CDRs). There is also an additional area of hypervariability on the β-chain (HV4) that does not normally contact antigen and, therefore, is not considered a CDR.

CDR3 is the main CDR responsible for recognizing processed antigen, although CDR1 of the alpha chain has also been shown to interact with the N-terminal part of the antigenic peptide, whereas CDR1 of the β-chain interacts with the C-terminal part of the peptide. CDR2 is thought to recognize the MHC. CDR4 of the β-chain is not thought to participate in antigen recognition, but has been shown to interact with superantigens.

The C domain of the TCR consists of short connecting sequences in which a cysteine residue forms disulfide bonds, which form a link between the two chains.

The “B-cell receptor” or “BCR” is a transmembrane receptor protein located on the outer surface of B cells. The BCR comprises a membrane-bound immunoglobulin (antibody) molecule of one isotype (IgD, IgM, IgA, IgG, or IgE) and a signal transduction moiety comprising a heterodimer Ig-α/Ig-β, bound together by disulfide bridges. Similar to the TCR, the V domain of the BCR α-chain and β-chain each have three hypervariable regions or CDRs, which form the antigen-binding site.

### Sequencing of Hypervariable Regions in TCR or BCR (TSO-Based)

When analyzing a B cell or T cell, it is often important to understand both its gene expression profile on a transcriptomic scale, and the BCR or TCR sequence that confers the antigen specificity. Even though each task alone can be accomplished using existing methods (e.g., single-cell gene expression profile can be readily achieved using DropSeq-like methods that feature oligo dT-based reverse transcription primer, and BCR/TCR sequencing can be achieved by replacing the oligo-dT with sequence complementary to the constant (C) region of the BCR/TCR), it is non-trivial to obtain both 3′ expression profile and BCR/TCR sequence. This example shows how to solve this problem using circularization-based DNA reorientation. T cell analysis is used as an example, but the same principle can be applied to B cell analysis. Some steps of this process are depicted in FIG. 4.

The mRNAs from greater than 100, 200, 500, 1000, 5000, 10,000, 20,000, etc. of T cells can be barcoded using a DropSeq-like approach. A modified inDrop™ can be used as the exemplary method. In this modified method, one can create greater than 1,000, 2,000, 5,000, 10,000, 20,000, etc. of water-in-oil droplets where there is only one T cell and one hydrogel bead, where the hydrogel bead embeds RT primers that carry the same cell barcode. The RT primer (401 of FIG. 4) can be constructed to have the following domains from 5′ to 3′ end: (a) a fixed-sequence domain DA which contains the PE1 site sequence (using the terminology of FIG. 2D of Klein et al. above); (b) a cell-barcode (CB) domain (i.e., ‘barcode1-W1-barcode2’ using the terminology of FIG. 2D of Klein et al. above); (c) an unique molecular identifier (UMI) domain; and (d) a polyT domain (PolyT). The T cells can be lysed in the droplets, releasing the mRNA content (including the mRNA molecules that encode the TCR which is depicted as 405 in FIG. 4 and has domains V (variable), D (diversity), J (joining), and C (constant)). The RT primers can then be released from the hydrogel bead by UV illumination. The RT primers then hybridize to the poly-A tail of the mRNA molecules and undergo reverse transcription to copy the mRNA including the mRNA encoding TCR (FIG. 4, Step 4.1).

After reverse transcription is completed, the reverse transcriptase can be heat-inactivated and the emulsion can be broken to pool all RT product. The reverse transcriptase may add a few C bases at the 3′ end of the first-strand cDNA. A template-switching oligo (TSO) which has a few G bases at the 3′ end can be added. The C bases at the 3′ end of the first-strand cDNA may pair with the G bases on the template-switching oligo and get extended using the template-switching oligo as a template (FIG. 4, Step 4.2). The sequence of the template-switching oligo (excluding the Gs at the 3′ end) is referred to as domain TS. The domain TS on the TSO may contain several deoxyuridine nucleotides, which can be cleaved using the USER™ enzyme mix (from New England Biolabs), causing the degradation of the domain TS (FIG. 4, Step 4.3).

Next, a primer comprising the TS sequence and a primer comprising the DA sequence can be used to amplify the first-strand cDNA (FIG. 4, Step 4.4). Additional sequences and modifications can be added to the 5′ end of these primers so that circularization can be performed using the method described in Section II, Step 2 above. This circularization process is depicted as Step 4.5 of FIG. 4, where the dashed lines represent a phosphodiester bond that link two segments of DNA.

Next, a new pair of primers (403 and 404 of FIG. 4) can be used as PCR primers to amplify the circular DNA ‘inside-out’ (FIG. 4, Step 4.6). Primer (403) has a domain C5* which is complementary to a segment of the C region close to the 5′ end of the C region. Primer (404) has a domain C3 that is identical to a segment of the C region close to the 3′ end of the C region. The 5′ ends of the primers (403) and (404) additionally contain domains DB* and DC, respectively, which provide additional primer binding sites which may facilitate downstream processing. This PCR amplification results in dsDNA molecules bookended by domains DC/DC* and DB/DB* (see the construct after Step 4.6 of FIG. 4).

Next, additional PCR steps can be performed to attach additional domains to the ends of the dsDNA (FIG. 4, Step 4.7), such as introducing domains necessary to perform NGS (e.g., P5 and P7) and sample indices (e.g., i5 or index read2 in Illumina platform). The location and sequences of C5 and C3 within the C region should be chosen so that (1) they cover conserved sequences shared by all TCR C domains of interest (such as TCR Beta C1 and TCR Beta C2), (2) they make the length of the final PCR product suitable for NGS, and (3) the distance between the J domain and the C5 domain is sufficiently short that the entire VDJ junction can be sequenced using the Illumina platform to identify the V, D, and J domains.

A primer essentially having the sequence of DA can be used as a sequencing primer to read the sequences of CB and UMI, and a primer essentially having the sequence of DB* can be used as a sequencing primer to read the sequences of domains J, D, and V. In some cases, the DA and DB* domains may essentially have the sequences of Rd2 and Rd1, respectively (Read2 and Read1, respectively, in the Illumina platform). And the step to read the sequences of CB and UMI can be essentially the same step of reading the i7 index (i.e., index read 1) in common Illumina sequencing run, except that more cycles may be used.

### Sequencing of Hypervariable Regions in TCR or BCR (V Gene Panel-Based)

To sequence paired TCRs or BCRs along with transcriptome, an alternative to using TSO is to use a panel of V gene primers for second strand synthesis. FIG. 8 shows an example of TCR-transcriptome co-sequencing using this strategy.

The design and production of primer 801 as well as Step 8.1 (reverse transcription in indexed droplets) can follow Klein et al above. After breaking the emulsion, an aliquot (hereby called the ‘TCR Aliquot’) representing ˜20% of the total volume of the aqueous phase can be used for V gene primer-based second strand synthesis (SSS) and PCR (Step 8.2). Each primer for SSS (named SSS Primer) has a sequence of [$zRd2|$V_Panel}, where $V_Panel is a variable sequence having many variants, each variant corresponding to a V gene of TCR alpha or beta chain.

To perform SSS of Step 8.2, the TCR Aliquot can be mixed with all the SSS Primers so that the final concentration of each SSS Primer is ˜5 nM, in the presence of ˜100 mM Na+ and ˜5 mM Mg++. The mixture will be heated to ˜60° C. for 5 hours to allow hybridization. Next, a thermostable DNA polymerase (e.g., Taq) along with dNTPs can be added to the mixture which allows the SSS Primers to extend on the template. This primer extension product can be SPRI-purified and named ‘SSS Product’.

The SSS Product can be PCR-amplified by primers having the sequence of $zRd1^{Δ} and $zRd2^{Δ} (see Table 4 for sequences). The sequence of these primers may also be truncated by 12- to 14-nt at the 3′ end to ensure specific amplification. This PCR amplification completes Step 8.2. Next, one may use primers having sequence [zX|zRd1^{Δ}} and [$X*|$Idx*|$zRd2^{Δ}} to perform PCR while introducing sample index (Step 8.3). Domain $Idx can be a 6- to 8-nt arbitrary which can serve as sample index. Domain $X may have the sequence shown in Table 5, and serve as the circularization domain. This PCR product can then be circularized (Step 8.4) using the method described in FIG. 2 and the associated text.

The circularized DNA can be amplified using primer 804 and 805 (Step 8.5), which essentially linearize and truncate the DNA. Primer 804 has the sequence [$P5|$C5*}. Primer 805 has the sequence of [$zP7|$C3}. This PCR product is suitable for standard HiSeq X or NovaSeq sequencing.

Sequencing Point Mutations Distant from the 3′ End

In some situations, it may be desired to analyze the transcriptome profile and mutation status of a cell simultaneously. For example, in tumor microenvironment there may be both tumor cells that carry a particular mutation and normal cells that do not carry such mutation. It may be desired to study the difference in transcriptome profiles between tumor cells and normal cells.

As an example, if tumor cells, but not normal cells, have K27M mutation in the H3F3A gene, one may process the sample using the strategy shown in FIG. 5. As in standard single-cell RNA-Seq methods, the tumor tissue can be disseminated into cell suspension. The cell suspension comprising both tumor cells and normal cells can be encapsulated in water-in-oil droplets with hydrogel beads embedding barcoded RT primer using the inDrop™ technology. The cells may be lysed in the droplets and the barcoded RT primer ((501) of FIG. 5, constructed the same way as (401) of FIG. 4) may be released form the hydrogel beads. The mRNAs from the cell can be reverse transcribed by the RT primer and the reverse transcriptase that is present in the droplet. During this step, the H3F3A mRNA that may carry the mutation may also be reverse transcribed, resulting in the first-strand cDNA that also carries the mutation. In FIGS. 5, (502) and (503) denote the position of the K27 mutation on the mRNA and the first-strand cDNA, respectively. The mRNA:cDNA duplex may be converted to double-stranded DNA (dsDNA) using, for example, a template-switching oligonucleotide (TSO) followed by PCR, the NEBNext™ Ultra II Kits, or other methods (FIG. 5, Step 5.2). An aliquot of the cDNA mixture can be taken out to test for the H3F3A status while another aliquot (or the rest of the cDNA mixture) can be used for single-cell transcriptome analysis.

To analyze H3F3A K27 mutation status, the cDNA can be PCR-amplified (FIG. 5, Step 5.3) using a pair of primer as follows: The first primer (504 of FIG. 5) contains a DU domain and a MU domain. The DU domain can be designed to facilitate circularization as described in Section II, Step 2 above. The MU domain can be designed to match the sequence shortly upstream of potential mutation site. The distance between the 3′ end of the DU domain and the potential mutation site may be between 1 and 50 bases. The second primer (505 of FIG. 5) can be designed to contain essentially the DA sequence. This PCR product can be circularized using the method described in FIG. 2. This circularized DNA may be further amplified using another set of primers (506 and 507 of FIG. 5). The first primer (506) contains domains DB* and MD5*. The sequence of MD5* is designed to be complementary to the DNA shortly downstream of the potential mutation site. The DB* sequence can be designed to facilitate sequencing in different platforms.

The second primer (507) contains a domain DC at the 5′ end and a domain MD3 at its 3′ end. The domain MD3 is designed to prime close to the 3′ end of the mRNA (excluding the polyA tail). The PCR amplification (FIG. 5, Step 5.5) can yield a linear dsDNA construct bookended by domains DC/DC* and DB/DB*. This PCR product can be further amplified with primers having additional domains to introduce new domains (such as P5, P7 and sample index domain i5 (index read2 in Illumina platform)) and the termini of the dsDNA (FIG. 5, Step 5.6). This final dsDNA can be sequenced using NGS.

### Quasi-Full Length Single Cell RNA-Seq

Most DropSeq-like ultra-high throughput scRNA-Seq methods only allow sequencing of the 3′ ends of the mRNAs. The methods described in Examples 1 and 2 show how one may obtain sequence upstream on the mRNA if one knows the sequence context in region of interest on the mRNA (e.g., the sequence in the C domain of TCR and the sequence around the potential point mutation site). However, in some embodiments one may wish to survey the full-length mRNA sequence in an exploratory or hypothesis-free fashion, without necessarily knowing the sequence context a priori. This example describes how one may achieve that with TeleLink™.

Synthesis of barcoded first-strand cDNA, where the barcode comprises both a cell barcode and a UMI domain, can be accomplished by insertion of an additional domain (domain DB) between the UMI and the poly-T region (domain PolyT) (see (601) of FIG. 6A). Domain DA is equivalent to domain P2 of FIG. 1, and may comprise the sequence of Rd2 as in Illumina sequencing platform. The sequence containing cell barcode (i.e., ‘barcode1-W1-barcode2’ using the terminology of FIG. 2D of Klein et al. above) is named domain CB. The purpose of domain DB is to provide a primer-binding site between the UMI and the poly-T region, which is equivalent to domain P3 of FIG. 1.

After the emulsion is broken, instead of using the CEL-Seq2 method for second-strand synthesis and amplification (as in the standard inDrop™ method), one may use the SMARTer (Switching Mechanism At 5′ End of RNA Transcript) method (e.g., using the SMARTer kit from Clontech Laboratories), which requires a template switching oligonucleotide (TSO). Such cDNA can be further amplified so that each initial mRNA molecule may be represented by multiple copies (shown as (601) of FIG. 6A). The amplified DNA may be further fragmented by the tagmentation reaction to introduce domain DC*/DC at the DNA break points. Domain DC is equivalent to domain X of FIG. 1. Since tagmentation is a random process, the multiple copies of the same cDNA (sharing the same CB and UMI) may be truncated at different positions (as in 602, 603 and 604 of FIG. 6A). As described in ‘Step 2’ of Section II, the domain DC*/DC may be designed to facilitate circularization (FIG. 6A, Step 6.2). The domain DA*/DA may be appended with additional sequences to facilitate the circularization. The circularized DNA may be subject to another round of tagmentation which introduces another domain: DD*/DD (FIG. 6B, top). Again, since the tagmentation is a random process, different copies may be broken at multiple positions. For simplicity, we name mRNA-derived sequence flanked by domains DC*/DC and DD*/DD on molecules (651), (652) and (653) on FIG. 6B domains TA*/TA, TB*/TB, and TC*/TC, respectively.

The DNA molecules that have undergone the second tagmentation reaction can be PCR-amplified using primers essentially having sequences DB* and DD (see the arrow in FIG. 6B). With this amplification, molecules (651), (652) and (653) may give rise to linear dsDNA molecules (654), (655), and (656), respectively. In FIG. 6C, new domains can be introduced into DNA molecule (658) to facilitate NGS (TA, TB, and TC are collectively referred to as TX for simplicity). To facilitate sequencing (using, for example, the Illumina platform), the domain DD of DNA molecule (659) can be essentially the Rd1 (Read1) domain, and the domain DA can be essentially the Rd2 (Read2) domain. Therefore, the typical ‘read 1’ of Illumina sequencing may yield the sequence of domain TX, and the typical ‘index read 1’ will yield the sequence of domains CB and UMI.

Overall, as schematically shown by (657) of FIG. 6B, multiple regions within the body of the mRNA may be sequenced (dashed arrows show the regions that can be sequenced). Importantly, these reads are linked with the same CB and UMI so that these reads can be identified to be from the same original mRNA molecule.

### EXAMPLE

### A. Example 1

### Successful Circularization While Minimizing Inter-Molecular Ligations

We use TCR sequences as model sequences to demonstrate the DNA circularization protocol. We prepared 2 dsDNA templates with the sequences called $TRA and $TRB, respectively, using Jurkat cell (Clone E6-1) cDNA and standard molecular biology methods. The sequences of $TRA and $TRB are listed in Table 7. We appended the GC-only domains (serving the purpose of the GC-only regions of 202 and 203 of FIG. 2, and the domains $X and $X* in FIG. 8) to both ends of $TRA by PCR-amplifying $TRA with primers $P01 and $P02. We name the sequence of the amplified product $TRA-X. Similarly, $TRB was amplified with primers $P01 and $P02 to obtain $TRB-X.

Next, the 3′-end GC-only regions on PCR product were chewed off using the Q5® High-Fidelity DNA Polymerase with the presence of dATP and dTTP only. We call these PCR product ‘digested’ TRA and TRB gene segments.

Next, we mixed digested TRA and TRB gene segments at the molar ratio of 1:100 at a series of concentrations (listed in FIG. 10), in which TRA represents the tested molecule while TRB represents all other molecules that potentially can form dimers or even oligomers with TRA in the same tube. Ligation was performed using the Instant Sticky-end Ligase Master Mix, and linear dsDNA without successful circularization was removed by Exonuclease V digestion.

To test the intra-molecular circularization efficiency versus inter-molecular ligation events, we designed primers having sequences $P03, $P04, $P05, and $P06 as shown in Table 6, targeting the 5′- and 3′-ends of the TRA and TRB gene segments as shown in FIG. 9 for qPCR quantification. Additional qPCR reactions were also carried out to quantify total TRA using primers having sequences $P07 and $P08. Ct values are listed below in FIG. 10.

Comparing Ct values of total TRA (row “P07+P08” in FIG. 10) and TRA circularization (row “P03+P04” in FIG. 10), almost all TRA gene segments detected after linear dsDNA digestion were circularized, while TRA-TRB (P04+P05 & P06+P03) inter-molecular ligation is about 64-fold less (different primers have been proved to have comparable amplification efficiencies, data not shown).

TABLE 6
Primer sequence used in Example 1.

| Primer | |
|---|---|
| Name | Sequences |
| $P01 | 5′-/Phos/CGCGCCCGCCATACTCTTTCCCTACACGACG |
| | CTCT-3′ |
| $P02 | 5′-GGCGGGCGCGATTTCGCCTTAGTGACTGGAGTTCAGA |
| | CGTG-3′ |
| $P03 | 5′-CGTCAGGTGGAAGGAGGTTTC-3′ |
| $P04 | 5′-GGCGTGTTGTATGTCCTGCTG-3′ |
| $P05 | 5′-CTGAGGGCTGGATCTTCAGAGTG-3′ |
| $P06 | 5′-GGACCTTAGCATGCCTAAGTGAC-3′ |
| $P07 | 5′-TCAAGCTGGTCGAGAAAAGCT-3′ |
| $P08 | 5′-ATTAAACCCGGCCACTTTCAG-3′ |

TABLE 7
Sequences of model substrates

| DNA | |
|---|---|
| Name | Sequences (top strand only) |
| $TRA | 5′- GTGACTGGAGTTCAGACGTGTGCTCTTCCGATCT |
| | AGAGTGAAACCTCCTTCCACCTGACGAAACCCTCAGCCCATATGA |
| | GCGACGCGGCTGAGTACTTCTGTGCTGTGAGTGANNGGGGTAC |
| | AGCAGTGCTTCCAAGATAATCTTTGGATCAGGGACCAGACTCAG |
| | CATCCGGCCAANATATCCAGAACCCTGACCCTGCCGTGTACCAGC |
| | TGAGAGACTCTAAATCCAGTGACAAGTCTGTCTGCCTATTCACCG |
| | ATTTTGATTCTCAAACAAATGTGTCACAAAGTAAGGATTCTGATG |
| | TGTATATCACAGACAAAACTGTGCTAGACATGAGGTCTATGGAC |
| | TTCAAGAGCAACAGTGCTGTGGCCTGGAGCAACAAATCTGACTT |
| | TGCATGTGCAAACGCCTTCAACAACAGCATTATTCCAGAAGACAC |
| | CTTCTTCCCCAGCCCAGAAAGTTCCTGTGATGTCAAGCTGGTCGA |
| | GAAAAGCTTTGAAACAGATACGAACCTAAACTTTCAAAACCTGTC |
| | AGTGATTGGGTTCCGAATCCTCCTCCTGAAAGTGGCCGGGTTTA |
| | ATCTGCTCATGACGCTGCGGCTGTGGTCCAGCTGAGATCTGCAA |
| | GATTGTAAGACAGCCTGTGCTCCCTCGCTCCTTCCTCTGCATTGC |
| | CCCTCTTCTCCCTCTCCAAACAGAGGGAACTCTCCTACCCCCAAG |
| | GAGGTGAAAGCTGCTACCACCTCTGTGCCCCCCCGGTAATGCCAC |
| | CAACTGGATCCTACCCGAATTTATGATTAAGATTGCTGAAGAGCT |
| | GCCAAACACTGCTGCCACCCCCTCTGTTCCCTTATTGCTGCTTGT |
| | CACTGCCTGACATTCACGGCAGAGGCAAGGCTGCTGCAGCCTCCC |
| | CTGGCTGTGCACATTCCCTCCTGCTCCCCAGAGACTGCCTCCGCC |
| | ATCCCACAGATGATGGATCTTCAGTGGGTTCTCTTGGGCTCTAGG |
| | TCCTGGAGAATGTTGTGAGGGGTTTATTTTTTTTTAATAGTGTTC |
| | ATAAAGAAATACATAGTATTCTTCTTCTCAAGACGTGGGGGGAA |
| | ATTATCTCATTATCGAGGCCCTGCTATGCTGTGTGTCTGGGCGTG |
| | TTGTATGTCCTGCTGCCGATGCCTTCATTAAAATGATTTGGAAAA |
| | AGATCGGAAGAGCGTCGTGTAGGGAAAG |
| | -3′ |
| $TRB | 5′- GTGACTGGAGTTCAGACGTGTGCTCTTCCGATCT |
| | CCACTCTGAAGATCCAGCCCTCAGAACCCAGGGACTCAGCTGTGT |
| | ACTTCTGTGCCAGCAGTTTAGCNGGGACAGGGGGCNCTAACTAT |
| | GGCTACACCTTCGGTTCGGGGACCAGGTTAACCGTTGTAGNAGG |
| | ACCTGAACAAGGTGTTCCCACCCGAGGTCGCTGTGTTTGAGCCA |
| | TCAGAAGCAGAGATCTCCCACACCCAAAAGGCCACACTGGTGTG |
| | CCTGGCCACAGGCTTCTTCCCTGACCACGTGGAGCTGAGCTGGT |
| | GGGTGAATGGGAAGGAGGTGCACAGTGGGGTCAGCACGGACC |
| | CGCAGCCCCTCAAGGAGCAGCCCGCCCTCAATGACTCCAGATACT |
| | GCCTGAGCAGCCGCCTGAGGGTCTCGGCCACCTTCTGGCAGAAC |
| | CCCCGCAACCACTTCCGCTGTCAAGTCCAGTTCTACGGGCTCTCG |
| | GAGAATGACGAGTGGACCCAGGATAGGGCCAAACCCGTCACCCA |
| | GATCGTCAGCGCCGAGGCCTGGGGTAGAGCAGACTGTGGCTTT |
| | ACCTCGGTGTCCTACCAGCAAGGGGTCCTGTCTGCCACCATCCTC |
| | TATGAGATCCTGCTAGGGAAGGCCACCCTGTATGCTGTGCTGGT |
| | CAGCGCCCTTGTGTTGATGGCCATGGTCAAGAGAAAGGATTTCT |
| | GAAGGCAGCCCTGGAAGTGGAGTTAGGAGCTTCTAACCCGTCAT |
| | GGTTTCAATACACATTCTTCTTTTGCCAGCGCTTCTGAAGAGCTG |
| | CTCTCACCTCTCTGCATCCCAATAGATATCCCCCTATGTGCATGC |
| | ACACCTGCACACTCACGGCTGAAATCTCCCTAACCCAGGGGGACC |
| | TTAGCATGCCTAAGTGACTAAACCAATAAAAATGTTCTGGTCTGG |
| | CCTGAAAAAAAAAAAAAAAAAAAAGATCGGAAGAGCGTCGTGTAG |
| | GGAAAG-3′ |

### Equivalents

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term “about” refers to a numeric value, including, for example, whole numbers, fractions, and percentages, whether or not explicitly indicated. The term “about” generally refers to a range of numerical values (e.g., +/−5-10% of the recited range) that one of ordinary skill in the art would consider equivalent to the recited value (e.g., having the same function or result). When terms such as “at least” and “about” precede a list of numerical values or ranges, the terms modify all of the values or ranges provided in the list. In some instances, the term “about” may include numerical values that are rounded to the nearest significant figure.

What is claimed is:

1. A method for generating truncated and barcoded nucleic acid molecules from at least two target polynucleotide sequences each from distinct biological particles comprising:
a. providing at least two heterogeneous pools of barcoded nucleic acid molecules each from a distinct biological particle, wherein each of the barcoded nucleic acid molecules comprise a target polynucleotide sequence and a barcode, wherein the barcode is unique to the distinct biological particle from which the barcoded nucleic acid molecule originated;
b. circularizing the barcoded nucleic acid molecules to obtain circular barcoded nucleic acid molecules; and
c. linearizing the circular barcoded nucleic acid molecules to obtain truncated and barcoded nucleic acid molecules comprising a truncated portion of the target polynucleotide sequence in the circular barcoded nucleic acid molecule and the barcode in the circular barcoded nucleic acid molecule.

2. The method of claim 1, further comprising amplifying the truncated barcoded nucleic acid molecules to obtain a barcoded amplified product comprising the barcode and the portion of the target polynucleotide sequence.

3. The method of claim 2, wherein the truncated nucleic acid molecules are amplified using primers capable of binding to the primer-binding sites.

4. The method of claim 2 or 3, wherein the barcoded amplified product comprises a length of equal to or less than 500 base pairs.

5. The method of claim 1, wherein the barcoded nucleic acid molecules further comprise at least one primer binding site.

6. The method of claim 1, further comprising introducing at least one primer-binding site to the truncated and barcoded nucleic acid molecules.

7. The method of any one of the claims 1 to 6, further comprising truncating the target polynucleotide sequence before circularizing the barcoded nucleic acid molecules.

8. The method of claim 7, further comprising ligating at least one additional domain to the truncated end of the barcoded nucleic acid molecule before circularizing the barcoded nucleic acid molecules.

9. The method of any one of the claims 1 to 8, further comprising ligating at least one additional domain to barcoded nucleic acid molecules before circularizing the barcoded nucleic acid molecules.

10. The method of any one of claims 1 to 9, wherein the barcoded nucleic acid molecule is DNA, RNA, or bisulfite-treated DNA.

11. The method of claim 10, wherein the target nucleic acid molecule is DNA.

12. The method of any one of the claims 1 to 11, wherein the target polynucleotide sequence is at least part of an engineered molecule that is used to engineer or probe the biological particle.

13. The method of any one of the claims 1 to 13, wherein the length of circular barcoded nucleic acid molecules is greater than 1 kb, 1.5 kb, 2 kb, 3 kb, 5 kb, or 10 kb.

14. The method of any one of the claims 1 to 13, wherein the distinct biological particles comprise cells, nuclei, or a cell cluster.

15. The method of claim 14, wherein the biological particles are cells.

16. The method of claim 15, wherein at least some of the cells are prokaryotic cells.

17. The method of claims 15 to 16, wherein at least some of the cells are eukaryotic cells.

18. The method of claims 15 to 17, wherein at least some of the cells are engineered with DNA, RNA or viral vectors that encode one or more biological agents that cause RNA-mediated gene knockdown, genome editing, transcriptional alteration, or epigenetic alteration.

19. The method of claim 18, wherein the one or more biological agents comprise one or more of siRNA, shRNA, miRNA, zinc finger domains, transcription activator-like effector (TALE), Cas9, RNA with CRISPR origin.

20. The method of claim 14, wherein the cell cluster comprises a T cell and an antigen presenting cell.

21. The method of claim 14, wherein the cell cluster comprises a cell that expresses an antigen-recognizing agent and a cell that expresses an antigen.

22. The method of claim 21, wherein the antigen-recognizing agent comprises an antigen-recognizing protein or an antigen-recognizing polynucleotide.

23. The method of claim 22, wherein the antigen-recognizing protein comprises an antibody, a functional antibody fragment, or a T cell receptor.

24. The method of any one of claims 20 to 23, wherein the antigen is complexed with a major histocompatibility complex (MHC) molecule.

25. The method of any one of the claims 1 to 24, wherein the target polynucleotide sequence comprises a partial or complete T cell receptor sequence, or a partial or complete B cell receptor sequence.

26. The method of any one of the claims 1 to 25, wherein the target polynucleotide sequence comprises a mutation.

27. The method of any one of the claims 1 to 26, wherein the target polynucleotide sequence comprises a transcription start site.

28. The method of any one of the claims 1 to 27, wherein the target polynucleotide sequence comprises a splicing junction.

29. A method for sequencing a target nucleic acid molecule, comprising sequencing the barcoded amplified product of any one of the claims 1 to 28.


SEQUENCE LISTING (28 sequences)

SEQ ID NO: 1 - DNA, 33 bp, Artificial Sequence
  Synthetic
acactctttccctacacgacgctcttccgatct

SEQ ID NO: 2 - DNA, 34 bp, Artificial Sequence
  Synthetic
  misc_feature (1)..(6): n is a, c, g, or t
  misc_feature (29)..(34): n is a, c, g, or t
nnnnnngagtgattgcttgtgacgccttnnnnnn

SEQ ID NO: 3 - DNA, 6 bp, Artificial Sequence
  Synthetic
  misc_feature (1)..(6): n is a, c, g, or t
nnnnnn

SEQ ID NO: 4 - DNA, 21 bp, Artificial Sequence
  Synthetic
ttttttttttttttttttttv

SEQ ID NO: 5 - DNA, 33 bp, Artificial Sequence
  Synthetic
gtgactggagttcagacgtgtgctcttccgatc

SEQ ID NO: 6 - DNA, 20 bp, Artificial Sequence
  Synthetic
acactctttccctacacgac

SEQ ID NO: 7 - DNA, 21 bp, Artificial Sequence
  Synthetic
gtgactggagttcagacgtgt

SEQ ID NO: 8 - DNA, 10 bp, Artificial Sequence
  Synthetic
ggcgggcgcg

SEQ ID NO: 9 - DNA, 22 bp, Artificial Sequence
  Synthetic
aatgatacggcgaccaccgaga

SEQ ID NO: 10 - DNA, 21 bp, Artificial Sequence
  Synthetic
ccgtgtaccagctgagagact

SEQ ID NO: 11 - DNA, 24 bp, Artificial Sequence
  Synthetic
caagcagaagacggcatacgagat

SEQ ID NO: 12 - DNA, 22 bp, Artificial Sequence
  Synthetic
ggatcttcagtgggttctcttg

SEQ ID NO: 13 - DNA, 35 bp, Artificial Sequence
  Synthetic
  misc_feature: Phos at 5′ end
cgcgcccgccatactctttccctacacgacgctct

SEQ ID NO: 14 - DNA, 41 bp, Artificial Sequence
  Synthetic
ggcgggcgcgatttcgccttagtgactggagttcagacgtg

SEQ ID NO: 15 - DNA, 21 bp, Artificial Sequence
  Synthetic
cgtcaggtggaaggaggtttc

SEQ ID NO: 16 - DNA, 21 bp, Artificial Sequence
  Synthetic
ggcgtgttgtatgtcctgctg

SEQ ID NO: 17 - DNA, 23 bp, Artificial Sequence
  Synthetic
ctgagggctggatcttcagagtg

SEQ ID NO: 18 - DNA, 23 bp, Artificial Sequence
  Synthetic
ggaccttagcatgcctaagtgac

SEQ ID NO: 19 - DNA, 21 bp, Artificial Sequence
  Synthetic
tcaagctggtcgagaaaagct

SEQ ID NO: 20 - DNA, 21 bp, Artificial Sequence
  Synthetic
attaaacccggccactttcag

SEQ ID NO: 21 - DNA, 1194 bp, Artificial Sequence
  Synthetic
  misc_feature (114)..(115): n is a, c, g, or t
  misc_feature (178)..(178): n is a, c, g, or t
gtgactggagttcagacgtgtgctcttccgatctagagtgaaacctccttccacctgacgaaaccctcagcccatatgagcgacgcggctgagtacttctgtgctgtgagtgannggggtacagcagtgcttccaagataatctttggatcagggaccagactcagcatccggccaanatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgagatctgcaagattgtaagacagcctgtgctccctcgctccttcctctgcattgcccctcttctccctctccaaacagagggaactctcctacccccaaggaggtgaaagctgctaccacctctgtgcccccccggtaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctggagaatgttgtgaggggtttatttttttttaatagtgttcataaagaaatacatagtattcttcttctcaagacgtggggggaaattatctcattatcgaggccctgctatgctgtgtgtctgggcgtgttgtatgtcctgctgccgatgccttcattaaaatgatttggaaaaaaaaaaaaaaaaaaaagatcggaagagcgtcgtgtagggaaag

SEQ ID NO: 22 - DNA, 970 bp, Artificial Sequence
  Synthetic
  misc_feature (102)..(102): n is a, c, g, or t
  misc_feature (115)..(115): n is a, c, g, or t
  misc_feature (164)..(164): n is a, c, g, or t
gtgactggagttcagacgtgtgctcttccgatctccactctgaagatccagccctcagaacccagggactcagctgtgtacttctgtgccagcagtttagcngggacagggggcnctaactatggctacaccttcggttcggggaccaggttaaccgttgtagnaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgaaggcagccctggaagtggagttaggagcttctaacccgtcatggtttcaatacacattcttcttttgccagcgcttctgaagagctgctctcacctctctgcatcccaatagatatccccctatgtgcatgcacacctgcacactcacggctgaaatctccctaacccagggggaccttagcatgcctaagtgactaaaccaataaaaatgttctggtctggcctgaaaaaaaaaaaaaaaaaaaagatcggaagagcgtcgtgtagggaaag

SEQ ID NO: 23 - DNA, 10 bp, Artificial Sequence
  Synthetic
cgcgcccgcc

SEQ ID NO: 24 - DNA, 13 bp, Artificial Sequence
  Synthetic
ggcgggcgcgatt

SEQ ID NO: 25 - DNA, 13 bp, Artificial Sequence
  Synthetic
aatcgcgcccgcc

SEQ ID NO: 26 - DNA, 13 bp, Artificial Sequence
  Synthetic
cgcgcccgccata

SEQ ID NO: 27 - DNA, 16 bp, Artificial Sequence
  Synthetic
aatcgcgcccgccata

SEQ ID NO: 28 - DNA, 13 bp, Artificial Sequence
  Synthetic
tatggcgggcgcg